(19) United States
(12) Patent Application Publication
(10) Pub. No.: US 2023/0292996 A1
(43) Pub. Date: Sep. 21, 2023

(54) LUMEN FLUID CONTROL AND ENDOSCOPE COUPLING ADAPTOR

(71) Applicant: OTTEK LTD, Nazareth (IL)

(72) Inventors: Yoav Lichtenstein, Nazareth (IL); David Hanuka, Nazareth (IL); Tamir Tal, Nazareth (IL)

(73) Assignee: OTTEK LTD, Nazareth (IL)

(21) Appl. No.: 18/189,035

(22) Filed: Mar. 23, 2023

Related U.S. Application Data
(63) Continuation-in-part of application No. PCT/IL2021/051148, filed on Sep. 22, 2021.
(60) Provisional application No. 63/329,498, filed on Apr. 11, 2022.

(30) Foreign Application Priority Data
Sep. 24, 2020 (IL) 277621

(51) Int. Cl. A61B 1/00; A61B 1/015

(52) U.S. Cl. CPC A61B 1/00131; A61B 1/015; A61B 1/00082

(57) ABSTRACT

Lumen fluid conducting and endoscope coupling adaptor for insertion through a lumen entry into a lumen of a patient, comprising: a proximal external body-recliner; a protruding muzzle coupled to the recliner which penetrates through the lumen entry into the proximal portion of the lumen; a collapsible clutch disposed at the distal end of the muzzle, configured to assume an expanded stat in the lumen for fixating the adaptor; a longitudinally extending endoscope bore allowing insertion and retraction of an endoscope through the adaptor; and a lumen fluids conduction arrangement, comprising: a longitudinally extending conduit in fluid communication with the endoscope bore; a fluid suction port in fluid communication with the conduit for releasing fluids from the lumen; a proximal seal for preventing outward leakage of the lumen fluids; and a lumen seal for preventing backflow of lumen fluids by sealing the gap between and protruding muzzle and the lumen.

### CROSS-REFERENCE TO RELATED APPLICATIONS

This application claims benefit of U.S. Provisional Application No. 63/329,498, filed Apr. 11, 2022, and is a continuation-in-part of PCT International Application No. PCT/IL2021/051148, filed Sep. 22, 2021, which claims benefit of U.S. Provisional Application No. 63/148,167, filed Feb. 11, 2021, and claims priority of Israeli Application No. 277621, filed Sep. 24, 2020, the entire contents of each of which are hereby incorporated by reference into the subject application.

### FIELD OF THE INVENTION

The present invention relates generally to insertion of objects and management of fluids within a lumen of a patient, and in particular to methods and adaptors for inserting medical instruments and managing fluids within a colon.

### BACKGROUND OF THE INVENTION

The diagnostic and therapeutic advantages conferred by direct examination of a body cavity and specifically of the gastrointestinal tract with a flexible endoscope, have made this method a standard procedure of modern medicine. One of the most common endoscopic procedures is a colonoscopy, which is performed for a wide variety of purposes, including diagnosis of cancer, determination of the source of gastrointestinal bleeding, viewing a site affected by inflammatory bowel disease, removing polyps, reducing volvulus and intussusception, and more.

In this procedure the endoscope is inserted and maneuvered through the entire length of the colon extending about 1.2-1.5 meters, and optimal maneuverability, stability and unobstructed vision of the endoscope camera are of highest importance.

### SUMMARY OF THE INVENTION

In accordance with an aspect of the invention there is provided a lumen fluid conducting and endoscope coupling adaptor for insertion through a lumen entry into a proximal portion of the lumen of a patient, and allowing insertion of an endoscope from a proximal end of the adaptor external to the body of the patient to a distal end of the adaptor internally disposed in the lumen. The adaptor includes: a proximal external body-recliner, sized to preclude insertion of the recliner into the lumen; an interim protruding muzzle coupled to the body-recliner, sized to allow penetration of the muzzle through the lumen entry into the proximal portion of the lumen; a distal collapsible intra-lumen clutch disposed at the distal end of the protruding muzzle, configured to assume a folded narrow state allowing insertion and retraction through the lumen entry and proximal portion of the lumen, and to assume an expanded state in the lumen, distally of the proximal portion, for fixating the adaptor; a longitudinally extending endoscope bore bored along the adaptor through the muzzle to allow insertion and retraction of the endoscope through the bore into the lumen; and a lumen fluids conducting arrangement, including: a longitudinally extending peripheral conduit bored along the adaptor through the muzzle, in fluid communication with the endoscope bore; a fluid suction port in fluid communication with the peripheral conduit for releasing lumen fluids entering the peripheral conduit from the lumen, the lumen fluids including liquids or liquids combined with solids; an irrigation nozzle, configured to spurt cleansing fluid through said adaptor; a proximal seal for preventing outward leakage of the lumen fluids, disposed proximally of the adaptor, configured to seal the peripheral conduit and around the endoscope when inserted in the endoscope bore; and a lumen seal for preventing backflow of the lumen fluids by sealing a gap between the protruding muzzle and the lumen when the protruding muzzle is inserted within the lumen. The collapsible intra-lumen clutch may include an inflatable balloon fixed to the clutch, wherein inflating of the balloon expands the clutch into the expanded state. The lumen seal may include an inflatable torus balloon, wherein the torus balloon is operational when inflated to prevent backflow of the lumen fluids by sealing the gap between the protruding muzzle and the lumen, when the adaptor is inserted within the lumen. The collapsible intra-lumen clutch may include a flapping peripheral flange which includes the lumen seal, wherein the flange is operational when in the expanded state to prevent backflow of the lumen fluids by sealing the gap between the muzzle and the lumen, when the adaptor is inserted within the lumen. The adaptor may be split into at least two fastenable shells, allowing splitting the endoscope bore and the proximal seal into an open state for facilitating disposition of the endoscope in the endoscope bore and the proximal seal, by allowing lateral laying of the endoscope therein before fastening of the fastenable shells into a closed state and facilitating insertion and retraction of the endoscope. The collapsible intra-lumen clutch may include an inflatable crescent balloon fixed to the clutch, such that inflating of the crescent balloon expands the clutch into the expanded state, and may further include a flapping peripheral flange, surrounding the distal end of the muzzle in a curl that overlaps a full circle and fixed to the crescent balloon, such that inflating of the crescent balloon expands the peripheral flange into an expanded state, and wherein overlapping of the peripheral flange tightly closes the crescent balloon into a full ring shape. The lumen seal may include an inflatable crescent balloon, operational when inflated to prevent backflow of the lumen fluids by sealing the gap between the muzzle and the lumen when inserted within the lumen. The collapsible clutch may include an inflatable balloon which is connected to an inflating tube, wherein the tip of which tube remains external to the lumen following insertion of the protruding muzzle, for allowing external control of inflating and deflating the inflatable balloon. The adaptor may include a cleaning rod featuring a distal cleaning tip, wherein the rod is sealingly inserted through the proximal seal and through the peripheral conduit, the rod extending longitudinally through the adaptor, and operational to allow insertion and retraction of the rod for physical break up and grinding of fecal debris and removal of clogging matter. The cleaning rod may include at least one hollow arm extending along the length of the cleaning rod, for allowing insertion and retraction of a longitudinal tube in the hollow arm. The endoscope bore may be in fluid communication with the suction port, and may include helical grooves operational to promote vortex flow of lumen fluids. The external body-recliner may include at least one of: a cushion connected to resilient members; a cushion connected to a ratchet featuring a toothed bar which is movable with respect to a snapping tooth-stopper; an external inflatable balloon; and a pulling strap fixed to the clutch, wherein the clutch is mobile along the length of the muzzle, for allowing adjusting of the longitudinal position of the clutch in relation to the lumen. The adaptor may include a lumen fluid suction unit in fluid communication with the suction port, for evacuation of fluids from the lumen, and an irrigation unit in fluid communication with the lumen, for streaming irrigation fluids into the lumen. The adaptor may further include an external fluid controller for controlling the operation of the lumen fluid suction unit and the irrigation unit, the external fluid controller including measuring sensors for measuring fluid flow, vacuum level at the suction port, and/or estimating the amount of cleansing fluid inside the patient's lumen. The lumen fluid suction unit may include a sewage canister for collecting evacuated lumen fluids, the irrigation unit may include an irrigation canister from which irrigation fluids are streamed into the lumen, and the measuring sensors may include weighing scales for measuring fluids quantity inside the sewage canister and/or the irrigation canister, allowing to estimate the amount of cleansing fluid remaining inside the patient's lumen. The lumen fluid suction unit and the irrigation unit may be powered by a common motor and mounted on a common axis of a dual head pump, allowing synchronized operation of the lumen fluid suction unit and the irrigation unit. The lumen fluid suction unit may further include an air tube in fluid communication with an evacuation tube communicating the lumen suction unit with the lumen, and the measuring sensors my include a pressure sensor configured to measure the pressure level in the evacuation tube, wherein when the pressure level in the evacuation tube drops below a predefined threshold, the controller may control air streaming means for streaming air into the evacuation tube via the air tube, for raising the air pressure in the evacuation tube without halting the operation of the lumen fluid suction unit. The adaptor may be externally attached to the patient's body using straps, puling wires and or tapes. The adaptor may further include a narrowing grommet, operational to be installed at the proximal end of the endoscope bore for sealingly accommodating an endoscope.

According to another aspect of the invention, there is provided a method for lumen fluid conducting and endoscope coupling, which includes the procedures of: disposing an endoscope into an adaptor, wherein the adaptor includes a longitudinally extending endoscope bore bored through the adaptor, and a proximal seal configured to surround the endoscope inserted in the bore, effecting blocking of fluid leakage from the proximal end of the adaptor; inserting the distal end of the adaptor into the proximal portion of the patient lumen, the adaptor including a distal intra-lumen collapsible clutch, an interim protruding muzzle and a proximal external body recliner, and continuing insertion of the adaptor until the external body recliner interfaces the patient body, wherein a distal lumen seal is operative to block leakage from a gap between the distal end of the muzzle and the lumen; expanding the distal collapsible clutch within the proximal portion of the lumen into an expanded configuration which presses against the lumen to fixate the adaptor to the body of the patient; allowing lumen fluids to drain through a longitudinally extending peripheral conduit bored along the adaptor through the muzzle, and to be released through a fluid suction port which is in fluid communication with the peripheral conduit, the lumen fluids including liquids or liquids combined with solids, wherein the proximal seal blocks the fluids from leaking from the proximal end of the adaptor; and spurting cleansing fluid through the adaptor from an irrigation nozzle. The fixating may include inflating a distal inflatable balloon fixed to the clutch, wherein the inflating of the balloon expands the collapsible intra-lumen clutch into the expanded state. The lumen sealing may include inflating of a distal inflatable torus balloon, wherein the torus balloon is operational when inflated to prevent backflow of the lumen fluids by sealing the gap between the protruding muzzle and the lumen when the adaptor is inserted within the lumen. The lumen sealing may include expanding a distal flapping peripheral flange operational as the lumen seal, wherein the flange is operational when in the expanded state to prevent backflow of the lumen fluids by sealing the gap between the muzzle and the lumen when the adaptor is inserted within the lumen. The fixating may include inflating an internal balloon connected to the clutch through an inflating tube in fluid communication with the balloon, wherein the tip of which tube remains external to the lumen following the inserting, for allowing external access for inflating and deflating the balloon. The draining of the lumen may include physically breaking up and grinding of fecal debris and other clogging matter using a cleaning rod featuring a distal cleaning tip, the rod sealingly inserted through the proximal seal and the peripheral conduit, wherein the grinding includes inserting and retracting of the rod. The draining of the lumen may include at least one of: (1) spurting of cleansing fluid from an irrigation nozzle extending through at least one hollow arm extending along the length of said cleaning rod, when said irrigation nozzle is inserted into the lumen; and (2) monitoring the grinding and the draining of the lumen using a camera inserted into the lumen through the at least one hollow arm. The method may further include adjusting the longitudinal dimensions of the adaptor to fit the proximal potion of the lumen by at least one of: leaning a proximal external cushion connected to resilient members upon the patient's body; leaning a proximal external cushion connected to a ratchet featuring a toothed bar which is 20 movable with respect to a snapping tooth-stopper upon the patient's body; leaning a proximal external inflatable balloon with a regulated degree of inflation upon the patient's body; and/or pulling the clutch along the muzzle with a pulling strap fixed to the clutch, wherein the clutch is mobile along the length of the muzzle. The draining of the lumen may include: actively suctioning lumen fluids through said suction port; irrigating the lumen with irrigation fluids; and controlling the suctioning and the irrigating by an external fluid controller. The method may further include measuring, calculating and/or controlling fluid flow and/or vacuum level at the suction port, and/or estimating the amount of cleansing fluid inside the lumen of the patient. The suctioning may include elevating the pressure in an evacuation tube communicating a lumen fluid suction unit with the lumen to an elevated pressure above a predefined threshold, without halting the operation of the lumen fluid suction unit, by measuring a pressure level in the evacuation tube and streaming air into the evacuation tube, when the pressure level drops beneath the predefined threshold, until the pressure is elevated above the predefined threshold. The disposing of an endoscope into an adaptor may further include installing a narrowing grommet at the proximal end of the endoscope bore to sealingly accommodate an endoscope.

### BRIEF DESCRIPTION OF THE DRAWINGS

The present invention will be understood and appreciated more fully from the following detailed description taken in conjunction with the drawings, in which:

FIG. 1 is a schematic side view illustration of an embodiment of a lumen fluid control and endoscope coupling adaptor, constructed and operative in accordance with the invention;

FIG. 2 is a schematic illustration of the adaptor of FIG. 1, as seen when rotated horizontally at 90°;

FIG. 3 is a schematic, cross-sectional illustration of the adaptor of FIG. 1, taken along line A-A denoted in FIG. 2;

FIG. 4 is a schematic illustration of the adaptor of FIG. 1 shown from top view;

FIG. 5 is a schematic illustration of the adaptor of FIG. 1 shown from bottom view;

FIG. 6 is a schematic illustration of the adaptor of FIG. 1 shown fixed on a patient's body;

FIGS. 7A to 7C illustrate successive stages of laterally disposing an endoscope on the adaptor of FIG. 1. FIG. 7A is a schematic illustration of the adaptor of FIG. 1 shown in an initial stage of an open/split configuration;

FIG. 7B is a schematic illustration of the adaptor of FIG. 7A shown in an interim stage of laterally disposing an endoscope into the adaptor when in its open/split configuration;

FIG. 7C is a schematic illustration of the adaptor of FIG. 7B shown in an operational stage where the adaptor is fastened about the endoscope;

FIGS. 8A to 8B illustrate the fastening of a splitable adaptor about an endoscope by means of a fastener. FIG. 8A is a schematic illustration of an embodiment of lumen fluid control and endoscope coupling adaptor, featuring a fastener in an open configuration, constructed and operative in accordance with the invention;

FIG. 8B is a schematic illustration of the adaptor of FIG. 8A, with the fastener in a closed configuration;

FIGS. 9A to 9D illustrate a means for narrowing or sealing the proximal end of the endoscope bore to accommodate endoscopes of varying diameters. FIG. 9A is a schematic illustration of an embodiment of a lumen fluid control and endoscope coupling adaptor, shown with the proximal side up, featuring a narrowing grommet and coupled with an endoscope, constructed and operative in accordance with the invention;

FIG. 9B is a schematic illustration of the adaptor of FIG. 9A shown without an endoscope;

FIG. 9C is a schematic illustration of an embodiment of a splitable narrowing grommet, constructed and operative in accordance with the invention;

FIG. 9D is a schematic illustration of the adaptor of FIG. 9A shown with the proximal side up, featuring a narrowing grommet and a sealing plug;

FIGS. 10A to 100 illustrate successive stages of insertion through the lumen entry and fixation of the adaptor coupled with an endoscope as shown in FIG. 7C, demonstrating the operation of a body recliner and a clutch of the adaptor. FIG. 10A is a schematic illustration of the adaptor in an initial stage positioned adjacently to the lumen entry;

FIG. 10B is a schematic illustration of the adaptor shown in an interim stage of insertion of its distal protruding muzzle into a proximal portion of the lumen, (with the clutch in a collapsed state), until encountering resistance by the body recliner;

FIG. 10C is a schematic illustration of the adaptor shown in a final stage of expanding of the distal clutch through inflation of its adjoined crescent balloon, thus fixating the adaptor and sealing the gap between lumen and adaptor;

FIGS. 11A to 11C show different aspects of the peripheral flapping flange of the adaptor of FIG. 1. FIG. 11A is a schematic illustration of the flange, shown in split-open configuration;

FIGS. 11B and 110 are schematic illustrations, from top view and side view, respectively, of the flange placed in the adaptor around the distal end of the muzzle and fixed to an inflatable crescent balloon;

FIGS. 12A to 12B illustrate a ribbed balloon. FIG. 12A is a schematic cross-sectional illustration of a ribbed balloon, constructed and operative in accordance with the invention;

FIG. 12B is a schematic illustration of the ribbed balloon of FIG. 12A shown from an above angle;

FIGS. 13A to 13C illustrate stages of the operation of the cleaning rod of FIG. 1. FIG. 13A is a schematic illustration of the adaptor from top-side angle, shown with the cleaning rod in distally extended position;

FIG. 13B is a schematic illustration of the adaptor from top-side angle, shown with the cleaning rod in proximally retracted position;

FIG. 13C is a schematic illustration of the cleaning rod;

FIGS. 14A to 14C illustrate a cleaning rod featuring a camera tube and an irrigation nozzle. FIG. 14A is a schematic illustration of an embodiment of a lumen fluid control and endoscope coupling adaptor, shown with the proximal side up, featuring a cleaning rod coupled with a camera tube and an irrigation nozzle, constructed and operative in accordance with the invention;

FIG. 14B is a schematic illustration of the adaptor and the cleaning rod of FIG. 14A, shown from above;

FIG. 14C is a schematic illustration of the adaptor and the cleaning rod of FIG. 14A, coupled with an endoscope;

FIGS. 15A to 15B show a cross section of the adaptor of FIG. 1 displaying components of a fluid conducting arrangement. FIG. 15A is a schematic illustration of the adaptor;

FIG. 15B is a schematic, cross-sectional illustration of the adaptor taken along line B-B denoted in FIG. 11A;

FIGS. 16A to 16D illustrate alternative fixating mechanisms of the body recliner of an embodiment of a lumen fluid control and endoscope coupling adaptor, constructed and operative in accordance with the invention. FIG. 16A is a schematic illustration of the adaptor, where the body recliner includes a cushion fixed to a ratchet;

FIG. 16B is a schematic illustration of the adaptor, where the body recliner includes an inflatable crescent balloon;

FIG. 16C is a schematic illustration of the adaptor, where a pulling strap fixed to the clutch serves to adjust and fix the longitudinal position of the adaptor;

FIG. 16D is a schematic illustration of the adaptor, where the body recliner includes a cushion fixed to a resilient member that is connected to a ratchet;

FIG. 17 is a schematic, cross-sectional illustration of an embodiment of a lumen fluid control and endoscope coupling adaptor and an external fluid management system, constructed and operative in accordance with the invention;

FIG. 18A is a schematic illustration of a balanced external fluid controller with pumps and dual weighing scales of an external fluid management system, constructed and operative in accordance with the invention;

FIG. 18B is a schematic cross-sectional illustration of an embodiment of a lumen fluid control and endoscope coupling adaptor and an external fluid management system, including a balanced external fluid controller, constructed and operative in accordance with the invention;

FIG. 19 is a schematic illustration of a further embodiment of a lumen fluid control and endoscope coupling adaptor, constructed and operative in accordance with the invention, featuring a lumen protecting element;

FIG. 20 is a schematic side view illustration of an embodiment of a lumen fluid control and endoscope coupling adaptor constructed and operative in accordance with the invention, featuring a distal clutch having an inflatable torus balloon;

FIGS. 21A to 21B illustrate an embodiment of a lumen fluid control and endoscope coupling adaptor constructed and operative in accordance with the invention, shown inserted into the patient's lumen and fixated using straps, tapes and wires attached to the patient's body. FIG. 21A is a schematic side view illustration of the adaptor attached to the patient's body using straps, wires and tape; and FIG. 21B is a schematic side view illustration of the adaptor attached to the patient's buttocks with tape;

FIG. 22 is a block diagram of a method for endoscope coupling and fluid conducting, operative in accordance with an embodiment of the present invention;

FIG. 23 is a block diagram of sub-routines or sub-procedures of steps 602 and 608 of the method of FIG. 22;

FIG. 24 is a block diagram of sub-routines or sub-procedures of steps 606 and 610 of the method of FIG. 22; and

FIG. 25 is a block diagram of sub-procedures of steps 602 and 604 of the method of FIG. 22.

### DETAILED DESCRIPTION OF THE DISCLOSED INVENTION

According to an embodiment of the present invention, there is provided an adaptor comprising fixating elements, an endoscope bore and a fluid-conducting arrangement, operational for fixating upon a patient's anus. The adaptor comprises a proximal external body-recliner, an interim protruding muzzle, operational for penetrating a lumen, a distal intra-lumen collapsible clutch and an endoscope bore bored along the length of the adaptor. Following insertion through the lumen entry and fixation of the adaptor within the lumen by expanding the collapsible clutch, and after insertion of an endoscope into the endoscope bore, preceding or following the initial adaptor anchorage, cleansing fluid is introduced into the patient's lumen through the endoscope. The term “endoscope”, and any variations thereof, as used herein refers to any type of probe or elongated instrument suitable to be inserted within a lumen. The fluid-conducting arrangement of the adaptor includes a peripheral conduit bored along the length of the adaptor, adjacent and in fluid communication with the endoscope bore; a fluid suction port in fluid communication with the peripheral conduit and the endoscope bore; and a proximal seal and a lumen seal, sealing the proximal outlet of the adaptor and the gap between the lumen and the adaptor, respectively. After distally streaming the fluids into the lumen via the endoscope, the fluid conducting arrangement leads the return flow of the streamed fluids mainly into the peripheral conduit and partly via the gap between the endoscope and the endoscope bore in which the endoscope is disposed, and out through the suction port, disposing of the fluids and preventing unpleasant leakage.

The lumen fluid conducting and endoscope coupling adaptor includes a proximal body-recliner which externally interfaces the patient's body; an interim protruding muzzle; and a distal collapsible internal clutch (the terms “intra-lumen” and “internal” in context of the clutch are used interchangeably and have the same meaning). Through the length of the adaptor runs a longitudinally extending endoscope bore through which an endoscope may be inserted into the lumen and retracted therefrom. The adaptor further includes a fluid conducting arrangement. The arrangement includes a longitudinally extending peripheral conduit, allowing lumen fluids to drain from the lumen; a proximal seal sealing the adaptor's internal volume while allowing the endoscope to pass through it without compromising the sealing; a lumen seal, sealing the gap between the lumen and the inserted protruding muzzle; and a suction port that is in fluid communication with the peripheral conduit and the endoscope bore, for releasing lumen fluids entering the peripheral conduit from the lumen. External fluids can be entered into the arrangement via the endoscope which can conduct these fluids directly into the lumen.

The adaptor may be splitable where two (or more) fastenable shells allow splitting the endoscope bore, the proximal seal, and the clutch into an open state suitable for lateral laying of the endoscope onto the endoscope bore. Thereafter, the shells and the clutch are fastened to encompass the endoscope in the bore.

Upon insertion of the protruding muzzle through the lumen entry and into a proximal portion of the lumen, the distal collapsible clutch expands to allow fixation of the adaptor. According to an embodiment of the invention, especially when the adaptor is splitable, the clutch may comprise an inflatable crescent balloon fixed to a peripheral overlap flapping flange, such that the crescent balloon and the flange are also splitable. Upon inflation, the balloon expands the flapping flange into an open operational clutch state and together with the flapping flange serves also as the lumen seal. The torus balloon and the crescent balloon may be inflated following insertion into the lumen by means of an inflating tube, the tip of which remains external to the lumen.

The protruding muzzle is inserted until the external recliner interfaces the patient's body, and following the expansion of the collapsible clutch, the recliner is adjusted to longitudinally fit the length of the proximal portion of the lumen. This adjustment can be effected by several means, such as: a cushion fixed to resilient members, a cushion fixed to a ratchet mechanism, a cushion fixed to resilient members and a ratchet mechanism, an inflatable external balloon and/or a pulling strap. The adaptor may be attached to the patient's body using tapes, straps, and or pulling wires.

Following fixation and stabilization of the adaptor coupled with the endoscope, irrigation fluids may be introduced into the lumen through the endoscope. The gap between the lumen and the adaptor having been sealed, the backflow of the fluids must necessarily drain through the peripheral conduit and the gap between the endoscope and the endoscope bore. With the proximal seal blocking the proximal end of the adaptor, the fluids flow out through the suction port. In order to improve fluid drainage, an lumen fluid suction unit may be in fluid connection with the suction port, for applying suction on the fluids in the lumen via the suction port. According to an embodiment of the invention, the suction unit may include a vacuum suction pump, disposed in fluid communication to a sewage canister or bag, where the pump may apply a suction force to the lumen fluids, through the suction port. Sensors such as weighing scales connected to a control unit may be used to measure the amount of fluids pumped out/drained via the suction port and the amount of fluids remaining in an irrigation canister from which fluids are pumped into the lumen, to estimate the amount of cleansing fluid remaining inside the patient's body. A sensor such as a pressure sensor connected to the control unit may be used to measure and control the vacuum level present at or applied to the suction port. A sensor such as a fluid flow sensor connected to the control unit may also be used to measure the fluid flow out of the suction port. Accompanying flushing of the lumen, the adaptor may include a cleaning rod inserted in the peripheral conduit and operational to grind fecal debris and other clogging matter by distally extending and retracting. The cleaning rod may include a distal irrigation nozzle and/or a camera to monitor the cleansing process.

The terms “irrigation fluid” and “cleansing fluid” are used herein interchangeably, and refer to a fluid, such as water, that is introduced into the lumen for irrigation of the lumen and flushing out of fecal debris or other clogging matter residing within the lumen.

The term “lumen fluids” as used herein refers to fluids which reside within the lumen, being of a liquid substance or a liquid mixed with solids, and including, for example, digestive juices, fecal debris, irrigation fluid which has been inserted into the lumen, and the like.

The terms “up”, “upwards”, and “above” as used herein refer to the distal direction or side of a lumen fluid irrigation and endoscope coupling adaptor, i.e., the side that is first inserted into a patient's lumen. The terms “down”, “downwards”, “bottom” and “bellow” as used herein refer to the proximal direction or side of a lumen fluid irrigation and endoscope coupling adaptor, i.e., the side that remains external to a patient's lumen.

Reference is now made to the Figures, in which like numbers designate like parts.

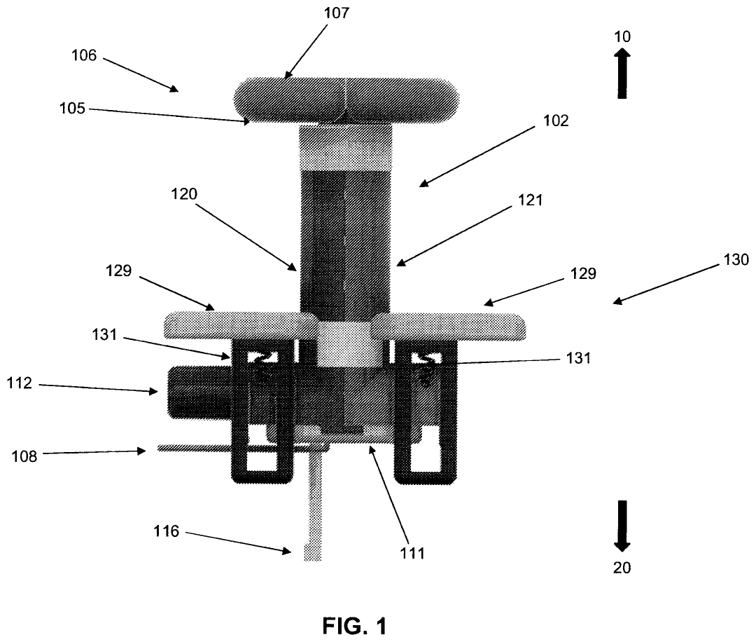

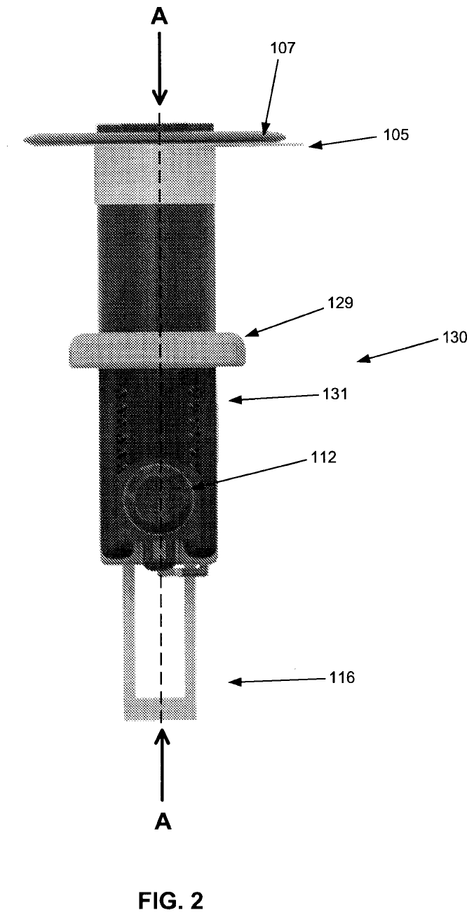

FIG. 1 is a schematic side view illustration of an embodiment of a lumen fluid control and endoscope coupling adaptor 100, constructed and operative in accordance with the invention. Reference is also made to FIG. 2 which is a schematic illustration of adaptor 100 as seen when rotated horizontally at 90°. Distal arrow 10 and proximal arrow 20 specify the distal and proximal sides of adaptor 100, respectively. Adaptor 100 includes proximal external body-recliner 130 comprising cushion 129 connected to resilient members 131, sized to preclude insertion of the recliner into the lumen; interim protruding muzzle 102, coupled to body-recliner 130, and sized so as to penetrate through lumen entry 150 into proximal portion of the lumen 152; and distal collapsible intra-lumen clutch 106, disposed at the distal end of protruding muzzle 102, and configured to assume a folded narrow state allowing insertion and retraction through lumen entry 150 and proximal portion of the lumen 152, and to assume an expanded state in the lumen, distally of proximal portion of the lumen 152, for fixating adaptor 100. Collapsible clutch 106 includes flapping peripheral flange 105 upon whose diameter is fixed to inflatable crescent balloon 107 which serves as an optional mechanism to expand collapsible clutch 106. An inflatable balloon may be used as an expansion mechanism for alternative embodiments of clutch 106. Clutch 106 is shown in its expanded state, with crescent balloon 107 in an inflated state. Crescent Balloon 107 is in fluid communication with inflating tube 108. The tip of tube 108 remains external to the lumen following insertion of protruding muzzle 102 into the lumen, and therefore allows external control of inflating and deflating crescent balloon 107 or any other internal balloon operational to expand clutch 106, following insertion of adaptor 100 into the lumen.

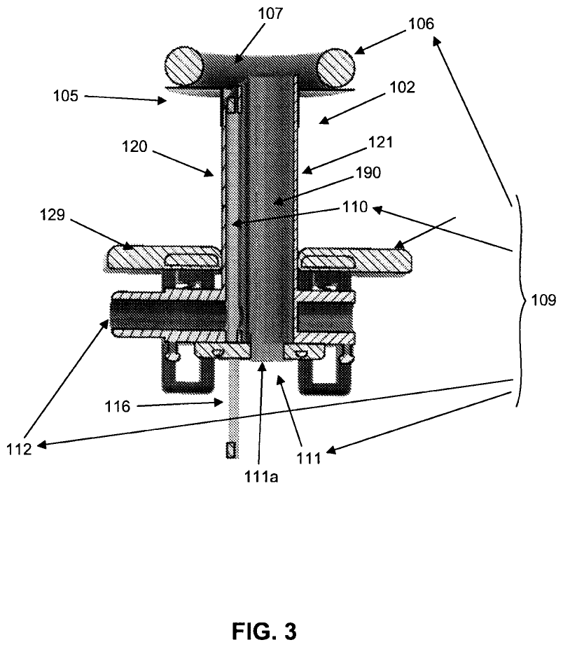

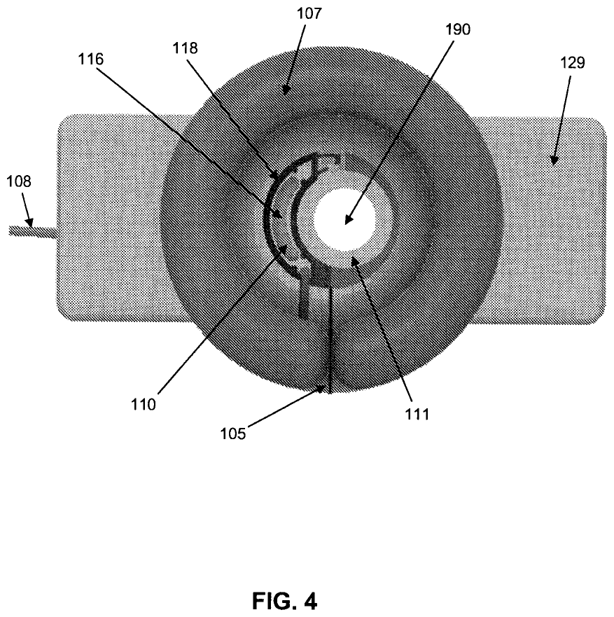

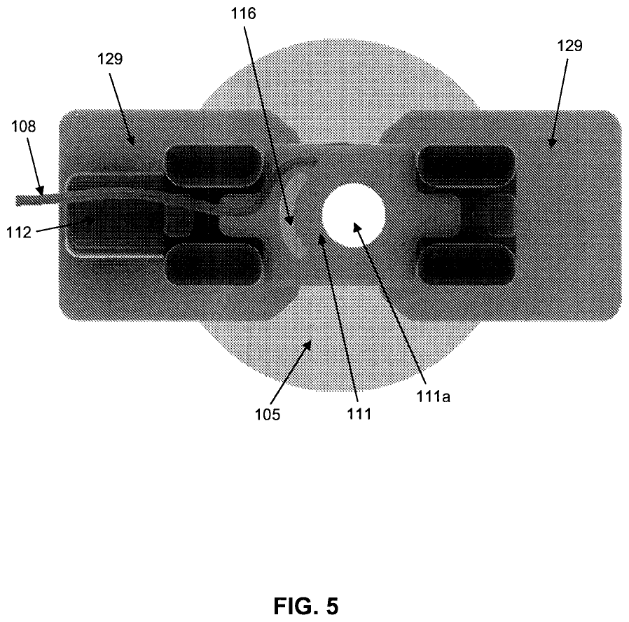

Reference is now also made to FIG. 3 which is a schematic, cross-sectional illustration of the adaptor of FIG. 1, taken along line A-A denoted in FIG. 2, to FIG. 4 which is a schematic illustration of adaptor 100 shown from top view, and to FIG. 5, which is a schematic illustration of adaptor 100 shown from bottom view. Through adaptor 100, peripheral conduit 110 and endoscope bore 190 are bored and are longitudinally extending along adaptor 100 through muzzle 102. The term “peripheral conduit” as used herein may refer to any portion of a lumen fluid control and endoscope coupling adaptor into which lumen fluids may flow from the lumen, and which is in fluid communication with a suction port, allowing the lumen fluids to be evacuated through the suction port. With reference to the Figures, an exemplary “peripheral conduit 110” is shown, which is an independent bore bored along adaptor 100. However, this is not meant to limit the definition of the term “peripheral conduit”, and, for example, a portion of endoscope bore 190 which is not occupied by an endoscope and into which lumen fluids may flow to exit through the suction port may also be considered a “peripheral conduit”.

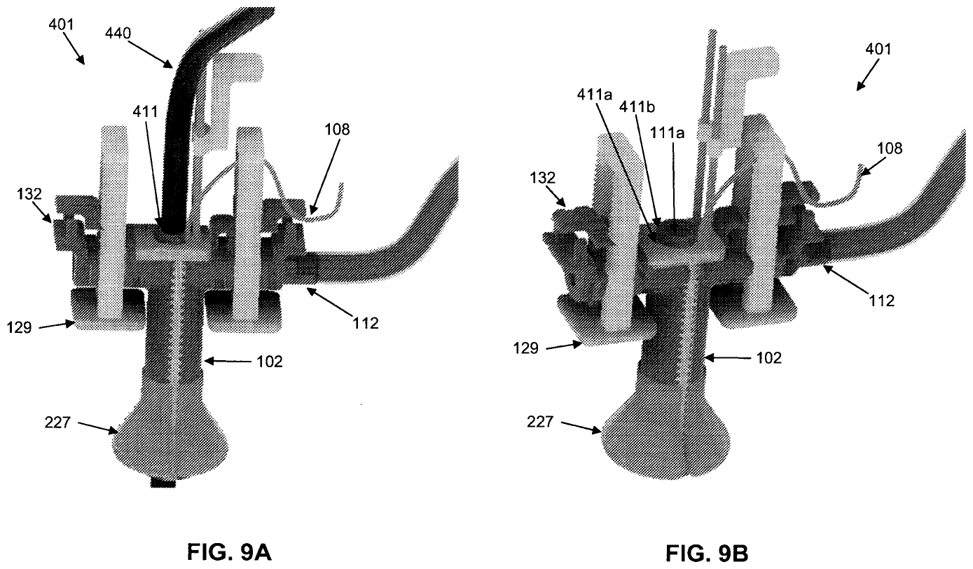

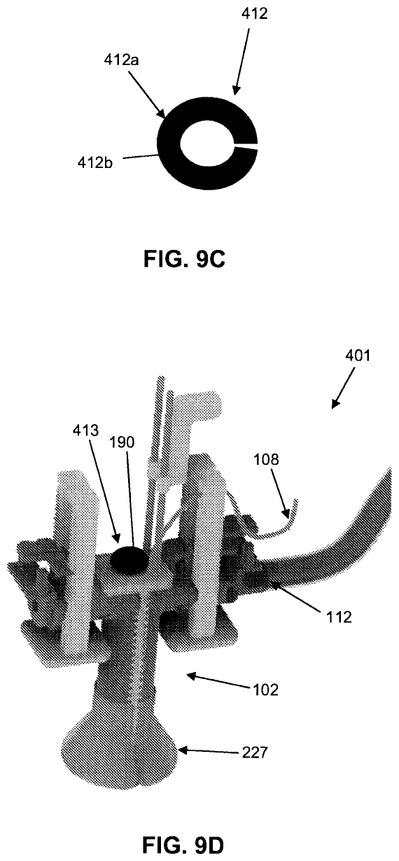

At the proximal end of peripheral conduit 110 and endoscope bore 190 resides proximal seal 111 for preventing outward leakage of the lumen fluids, configured to seal peripheral conduit 110 and around endoscope 140 when inserted in endoscope bore 190. The part of proximal seal 111 residing at the proximal end of endoscope bore 190 may be shaped so as to cover endoscope bore 190 while leaving a proximal opening 111a through which endoscope 140 may be sealingly inserted into endoscope bore 190. Peripheral conduit 110 and bore 190 are in fluid communication with fluid suction port 112, positioned distally to seal 111, serving as an outlet for releasing lumen fluids that enter conduit 110 and bore 190 from the lumen. Clutch 106, including peripheral flapping flange 105 and crescent balloon 107, serves in this embodiment of the invention also as a lumen seal (as further explained in reference to FIGS. 9A-9C), sealing the gap between protruding muzzle 102 and the inside of the lumen, for ensuring that the lumen fluids drain from the lumen through peripheral conduit 110 (and possibly through endoscope bore 190) out of suction port 112. Peripheral conduit 110, proximal seal 111, suction port 112, and clutch 106 as a lumen seal together make up lumen fluids conducting arrangement 109, which is in charge of directing the backflow of lumen fluids to a designated area and preventing their uncontrolled leakage.

Adaptor 100 further includes cleaning rod 116, sealingly inserted through proximal seal 111 and peripheral conduit 110 and extending longitudinally through adaptor 100, operational by extension and retraction to physically grind and break up fecal debris or remove clogging matter in a lumen. The body of adaptor 100 is made up of two fastenable shells 120-121, allowing splitting adaptor 100, flapping flange 105, endoscope bore 190 and proximal seal 111 into an open state, facilitating disposition of endoscope 140 in endoscope bore 190 and proximal seal 111 by allowing lateral laying of endoscope 140 therein and fastening of shells 120-121 and flange 105 thereof.

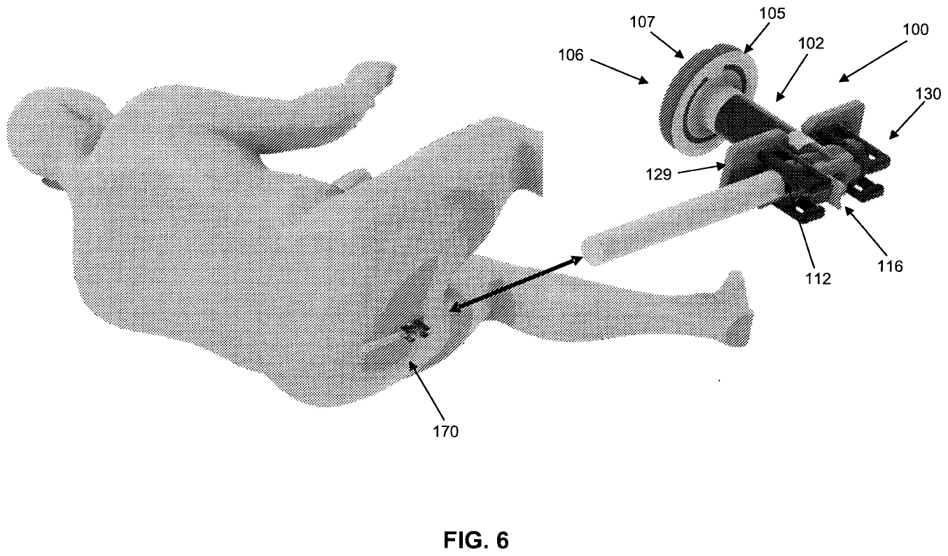

Reference is now made also to FIG. 6, which is a schematic illustration of adaptor 100 fixed on patient's body 170. Protruding muzzle 102 with distal clutch 106 is inserted through the patient's anus until recliner 130 interfaces with the patient's body 170, whereupon distal clutch 106 is expanded into its flange-like configuration and fixates adaptor 100.

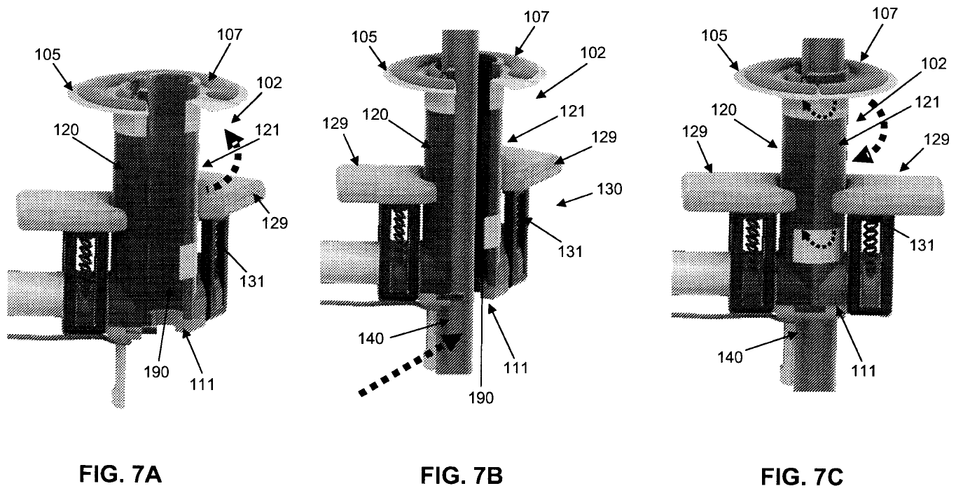

Reference is now also made to FIGS. 7A to 7C which illustrate successive stages of laterally disposing endoscope 140 within adaptor 100. FIG. 7A is a schematic illustration of adaptor 100 shown in an initial stage of an open/split configuration where shells 120-121 are separated along one side, splitting adaptor 100, flapping peripheral flange 105, endoscope bore 190 and proximal seal 111. This allows laterally laying of endoscope 140 within endoscope bore 190 and proximal seal 111 (as is also explained with reference to FIG. 7B). FIG. 7B is a schematic illustration of adaptor 100 shown in an interim stage of laterally disposing endoscope 140 into adaptor 100 when in its open/split configuration. FIG. 7C is a schematic illustration of adaptor 100 shown in an operational stage where adaptor 100 is fastened about endoscope 140. Adaptor 100 is fastened by re-adjoining shells 120-121 and assembling protruding muzzle 102 into a configuration operational to be inserted into the patient's proximal portion of the lumen 152. Adaptor 100 may be inserted into proximal portion of the lumen 152 before coupling with endoscope 140, and endoscope 140 may be inserted consequently.

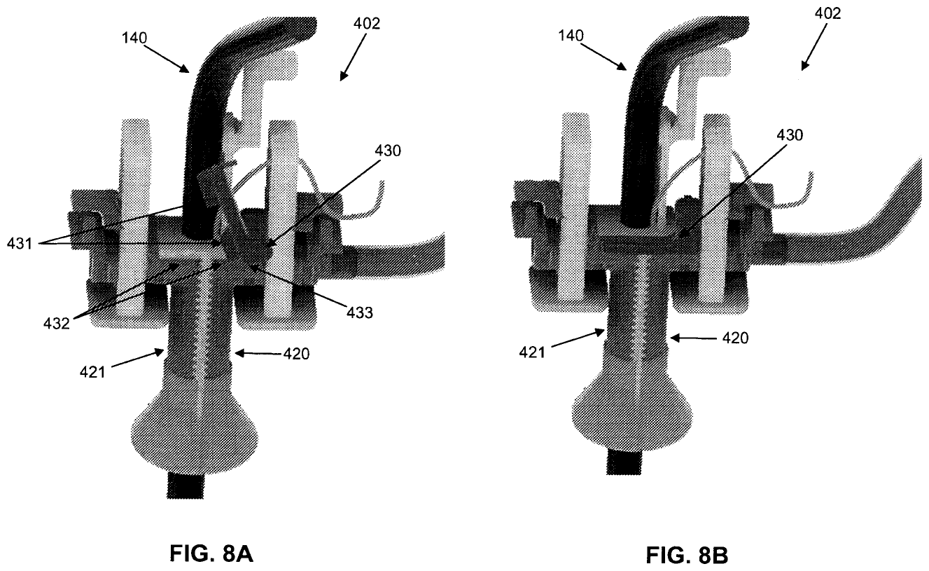

Reference is now also made to FIGS. 8A and 8B, which illustrate the fastening of fastenable shells 420, 421 of an adaptor 402 about endoscope 140 by means of a fastener 430. FIG. 8A is a schematic illustration of a lumen fluid control and endoscope coupling adaptor 402, featuring fastener 430 in an open configuration, constructed and operative in accordance with the invention. Fastener 430 features at least one alignment pin 431. Fastenable shells 420, 421 feature at least one alignment groove 432 corresponding to alignment pin 431. Fastener 430 may be connected to one of fastenable shells 420, 421 such as to allow fastener 430 to alternate between an open configuration, i.e., a configuration allowing splitting of fastenable shells 420, 421, and a closed configuration, i.e., a configuration which sealingly fastens fastenable shells 420, 421. For example, fastener 430 may be coupled to one of shells 420 or 421 through swinging axis 433, allowing fastener 430 to be raised into a vertical position when in an open configuration and lowered into a horizontal position when in a closed configuration. FIG. 8B is a schematic illustration of adaptor 402, with fastener 430 in a closed configuration. In order to securely fasten shells 420-421, usually about endoscope 140, at least one alignment pin 431 may be wedged into at least one alignment groove 432 and secured in place, thus maintaining shells 420-421 in a tightly fastened state. Fastener 430 may alternatively include a latch, a bolt, a tightening strap, or another type of fastening mechanism.

A wide range of endoscopic instruments known in the art display a variety of diameters with channels ranging from about 1.2 mm to about 6.0 mm, each of them specially suited for specific therapeutic procedures. In order to sealingly accommodate this variety of endoscopes in a single lumen fluid control and endoscope coupling adaptor, a means is needed for fitting the proximal end of endoscope bore 190 to the various endoscopes.

Reference is now made to FIGS. 9A to 9D, which illustrate a means for narrowing or sealing the proximal end of endoscope bore 190 to accommodate endoscopes of varying diameters. FIG. 9A is a schematic illustration of an embodiment of a lumen fluid control and endoscope coupling adaptor 401 shown with the proximal side up, featuring a narrowing grommet 411 and coupled with an endoscope 440, constructed and operative in accordance with the invention. Reference is also made to FIG. 9B which is a schematic illustration of adaptor 401 shown without an endoscope. Grommet 411 is installed at the proximal side of endoscope bore 190. Grommet 411 may be inserted within proximal opening 111a to be level and continuous with proximal seal 111, or it may be attached proximally to seal 111. Grommet 411 is ring-shaped and has an outer circumference 411a and an inner circumference 411b. Outer circumference 411a may be designed to fit tightly into proximal opening 111a of proximal seal 111 if grommet 411 is inserted within proximal opening 111a, or may be larger if grommet 411 is externally attached to proximal seal 111 of endoscope bore 190. The dimensions of inner circumference 411b may be adapted to sealingly accommodate a specific endoscope diameter which is smaller than the diameter of proximal opening 111a, for preventing leakage of lumen fluids from the proximal side of endoscope bore 190 and allowing full functioning of adaptor 401 when coupled with an endoscope of that specific diameter. Grommet 411 may be disposable, or may be reusable and permanently installed on adaptor 401.

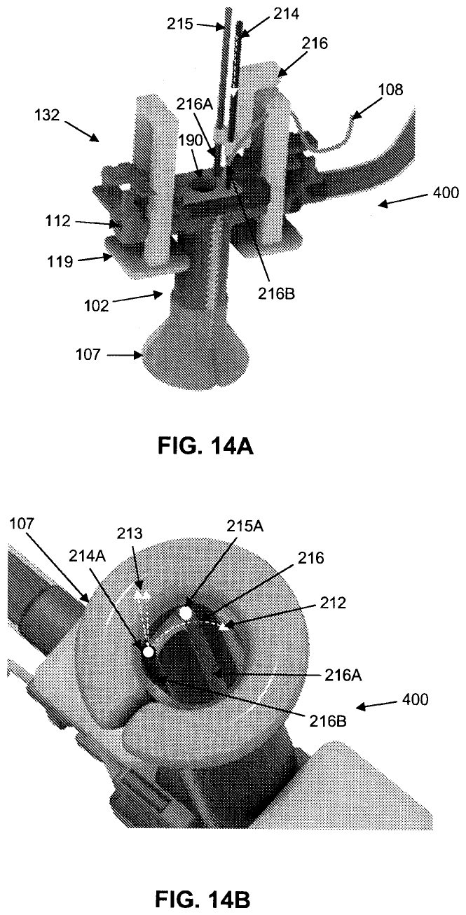

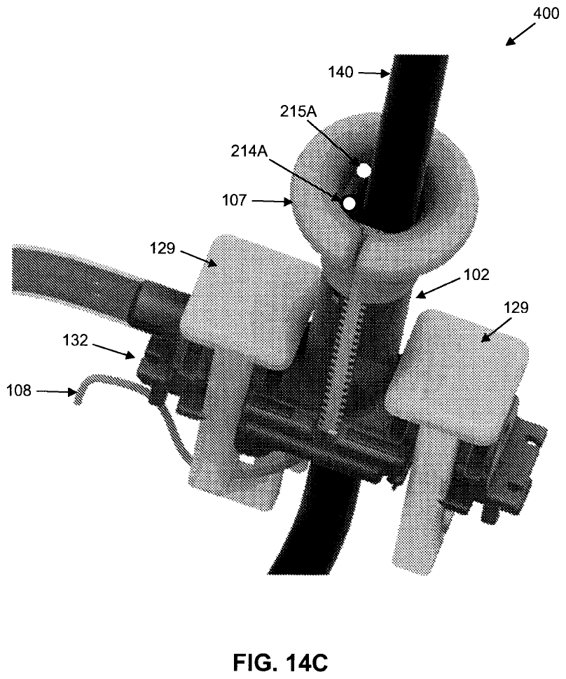

FIG. 9C is a schematic illustration of a splitable narrowing grommet 412. Splitable narrowing grommet 412 may be especially suited for laterally disposing an endoscope into a splitable lumen fluid control and endoscope coupling adaptor, as shown for example in FIGS. 7A to 7C with regard to adaptor 100. When adaptor 100 is split into an open configuration, grommet 412 may be installed at the proximal side of endoscope bore 190, either within proximal seal 111, or proximally or distally attached to proximal seal 111. An endoscope may then be laterally disposed within endoscope bore 190, proximal seal 111 and splitable narrowing grommet 412, all in a split open configuration, and adaptor 100 may subsequently be sealingly fastened about the endoscope. FIG. 9D is a schematic illustration of adaptor 401 shown with the proximal side up, featuring grommet 411 and a sealing plug 413. Sealing plug 413 may be disposed at the proximal end of bore 190 and proximally installed either on narrowing grommet 411 or directly on proximal seal 111, when grommet 411 is not installed. Sealing plug 413 is configured to block proximal opening 111a of proximal seal 111 when an endoscope is not disposed within proximal opening 111a and endoscope bore 190, to allow removal of lumen fluids through lumen fluids conducting arrangement 109. Lumen fluids which enter endoscope bore 190 from the distal end are prevented from leaking out of the proximal end of bore 190 by sealing plug 413, and are consequently conducted to exit adaptor 401 through suction port 112. In addition, as will be further explained with reference to FIGS. 14A to 14C, irrigation fluids and a camera may be introduced into the lumen independently of an endoscope, through cleaning rod 216, and cleaning rod 116/216 may also operate to grind fecal debris and other clogging matter residing in the lumen. In this manner, the lumen may be irrigated and cleared of clogging matter in the absence of an endoscope, and the lumen fluids will be evacuated only through suction port 112 due to sealing plug 413 blocking proximal opening 111a. Clearing the lumen in the absence of an endoscope may allow improved access to fecal debris and other clogging matter, and when an endoscope is subsequently inserted, may allow smoother advancement of the endoscope within the lumen.

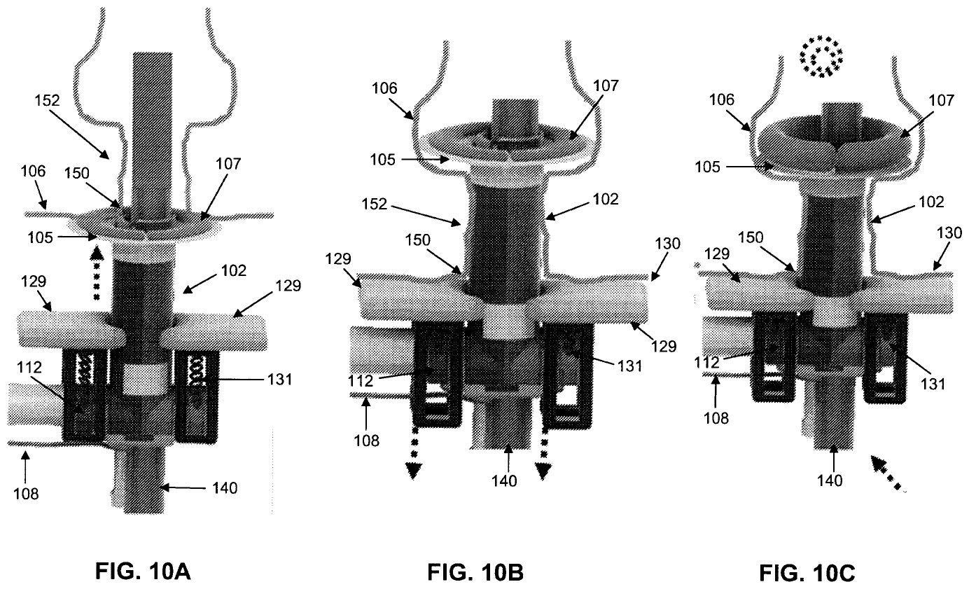

Reference is now also made to FIGS. 10A to 10C which illustrate successive stages of insertion through lumen entry 150 and fixation of adaptor 100 coupled with endoscope 140, demonstrating the operation of body recliner 130 and clutch 106 of adaptor 100. FIG. 10A is a schematic illustration of adaptor 100 coupled with endoscope 140 in an initial stage positioned adjacently to lumen-entry 150. Inflatable crescent balloon 107 is in a deflated state allowing flexibility of flapping flange 105 prior to insertion through lumen entry 150. FIG. 10B is a schematic illustration of adaptor 100 coupled with endoscope 140 shown in an interim stage of insertion of distal protruding muzzle 102 into proximal portion of the lumen 152, (with clutch 106 in a collapsed state) until encountering resistance by body-recliner 130. Recliner 130's resilient members 131 are compressed by patient's body 170 following insertion of distal protruding muzzle 102, and therefore exert a pulling force on adaptor 100 in the proximal direction. FIG. 10C is a schematic illustration of adaptor 100 coupled with endoscope 140 shown in a final stage of expanding of distal clutch 106 through inflation of adjoined crescent balloon 107, thus fixating adaptor 100 and sealing the gap between lumen 152 and adaptor 100. Inflatable crescent balloon 107 is blown up to its inflated state through inflating tube 108.

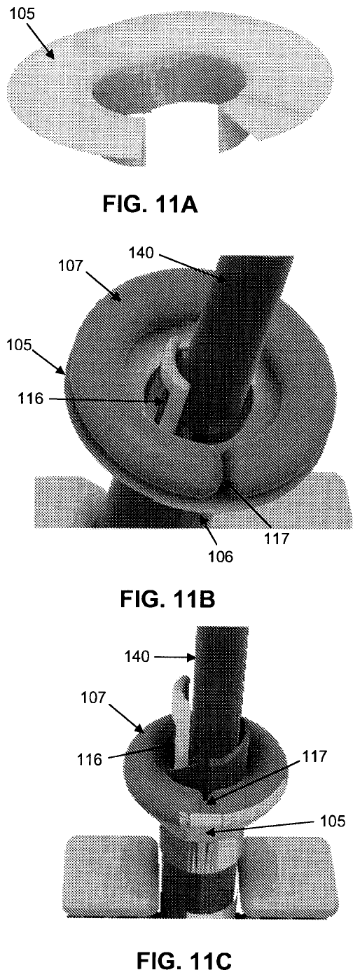

Reference is now also made to FIGS. 11A-11C, which show different aspects of peripheral flapping flange 105 of adaptor 100. FIG. 11A is a schematic illustration of flange 105, shown in split-open configuration. Emphasized is the splitable ring shape of flange 105 in a preferred design such that it overlappingly closes into a more than full circle, for example a round ring shape which curls at about 400° (360°+40°) about a full circle of 360°.

FIGS. 11B and 110 are schematic illustrations, from top view ad side view, respectively, of flange 105 placed around the distal end of muzzle 102 and fixed to inflatable crescent balloon 107 of adaptor 100. Gap 117 of balloon 107 and the split section of flange 105 are parallel and continual to the split of shells 120-121. Inflation of crescent balloon 107 induces swelling of flange 105 into an expanded state, and overlapping of flange 105 induces tightly closing crescent balloon 107 into a full ring shape (i.e., closing gap 117) allowing complete sealing of crescent balloon 107 which functions as a lumen seal, sealing the gap between adaptor 100 and proximal portion of the lumen 152.

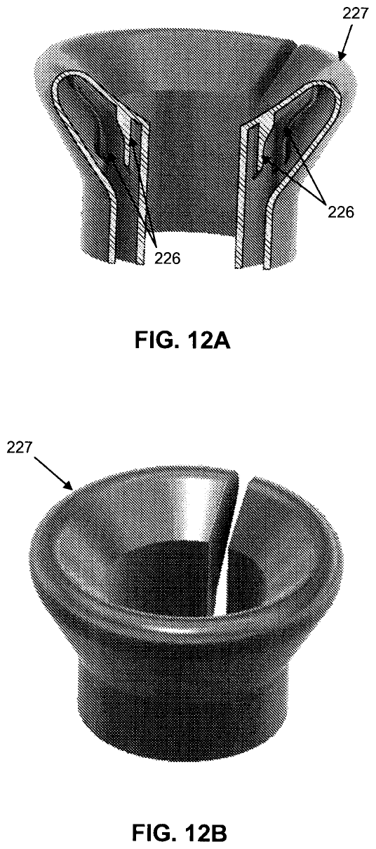

Reference is also made to FIGS. 12A to 12B, which illustrate a ribbed balloon 227. FIG. 12A is a schematic cross-sectional illustration of a ribbed balloon 227, constructed and operative in accordance with the present invention. FIG. 12B is a schematic illustration of ribbed balloon 227 shown from an above angle. Semi-rigid ribs 226 may be attached to the internal surface of ribbed balloon 227, and may be spaced at regular intervals along the circumference of ribbed balloon 227. The quantity of ribs 226 may be one or more, usually including at least two separate ribs 226, where their width and the spacing between them may vary. When ribbed balloon 227 is in a deflated state, ribs 226 do not affect the flexibility or collapsibility of ribbed balloon 227 (apart from by their own volume which is encompassed within balloon 227). However, when ribbed balloon 227 is inflated into an expanded state, i.e., following insertion into proximal portion of the lumen 152, semi-rigid ribs 226 mould the expansion of balloon 227 such that balloon 227 expands towards the lumen walls while limiting expansion towards endoscope bore 190. In this manner, expanded ribbed balloon 227 operates both as a clutch, firmly engaging the lumen walls, and as a lumen seal, sealing the gap between a distal protruding muzzle and the lumen wall. At the same time, the limited expansion of balloon 227 prevents balloon 227 from impeding lumen fluid evacuation through peripheral conduit 110 or interfering with the operation of endoscope 140. Ribbed balloon 227 may be a full closed ring, or may be crescent shaped such as to be splitable. A crescent shaped ribbed balloon 227 may be coupled to peripheral flapping flange 105, so as to tightly close into a full ring shape when inflated (as described with reference to FIGS. 11A to 110, regarding crescent balloon 107). Semi-rigid ribs 226 may alternatively be externally attached or otherwise positioned so as to mould the expansion of ribbed balloon 227.

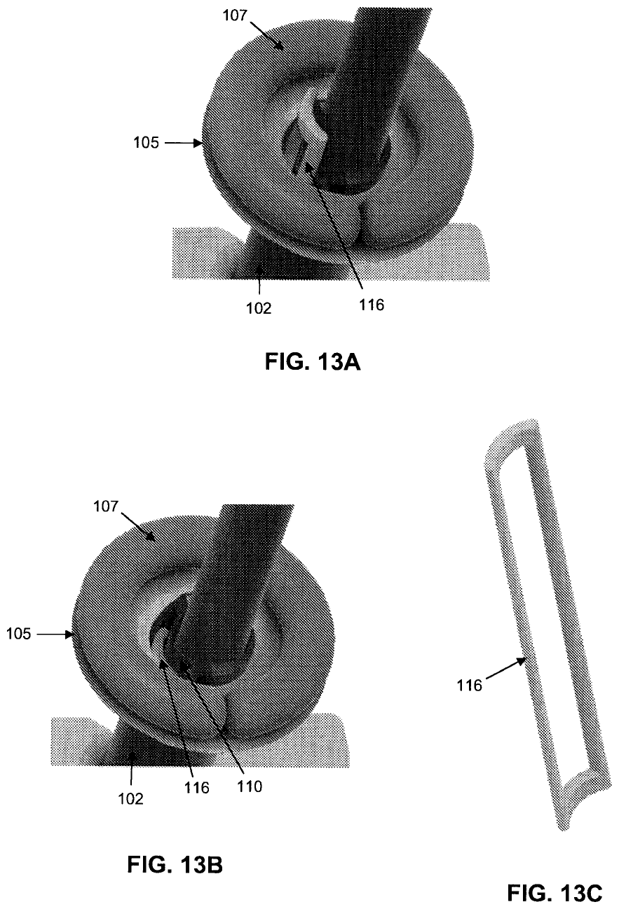

Reference is now made also to FIGS. 13A to 13C which illustrate stages of the operation of cleaning rod 116. FIG. 13A is a schematic illustration of adaptor 100 from top-side angle, shown with cleaning rod 116 in distally extended position Adaptor 100 is coupled with endoscope 140, and cleaning rod 116 is seen extending distally to flange 105 and crescent balloon 107, allowing physical contact and grinding of clogging matter residing in the lumen ahead of or peripherally to endoscope 140. FIG. 13B is a schematic illustration of adaptor 100 from top-side angle, shown with cleaning rod 116 in proximally retracted position. FIGS. 13A and 13B are otherwise identical, demonstrating the action of cleaning rod 116 through extension and retraction. Cleaning rod 116 extends longitudinally through peripheral conduit 110 from the distal end of adaptor 100 and out of the proximal end, allowing external manual extension and retraction of rod 116. FIG. 13C is a schematic illustration of cleaning rod 116, comprising, in its middle section, two arms 116a 116b.

Reference is also made to FIGS. 14A to 14C, which illustrate a cleaning rod 216 featuring a camera tube 215 and an irrigation nozzle 214. FIG. 14A is a schematic illustration of an embodiment of lumen fluid control and endoscope coupling adaptor 400, shown with the proximal side up, featuring cleaning rod 216 coupled with camera tube 215 and irrigation nozzle 214, constructed and operative in accordance with the invention. At least one irrigation nozzle 214 and/or at least one camera tube 215 may individually extend through a bore in any one of the arms (216a, 216b) of cleaning rod 216. Cleaning rod 216 may feature one or more hollow arms, and tubes of suitable diameter featuring instruments or accessories known in the art may extend through the hollow arms into the lumen. Irrigation nozzle 214 may be in fluid communication with an irrigation fluid pump (not shown) and may assist cleaning rod 216 in removal of clogging matter from the lumen by irrigating the clogging matter with irrigation fluid to dissolve the clogging matter or to detach it from the lumen wall. Camera tube 215 may also assist the operation of cleaning rod 216 by imaging the inside of the lumen and monitoring the cleaning and irrigation process, e.g., helping to identify the properties of clogging matter residing ahead in the lumen and allowing efficient handling of the clogging matter. Irrigation nozzle 214 and/or camera tube 215 may be maneuvered to be distally extracted into the lumen or proximally retracted, either in accordance with the extraction and retraction of cleaning rod 216 or independently thereof. Proximal external portions 214b, 215b of irrigation nozzle 214 and camera tube 215, respectively, reside outside the patient's body and allow manual extraction and retraction of distal tip 214a of irrigation nozzle 214 and camera head 215a of camera tube 215.

FIG. 14B is a schematic illustration of adaptor 400 and cleaning rod 216 shown with the proximal side up. Camera head 215a and distal tip 214a of irrigation nozzle 214 are shown at the distal end or arms 216a and 216b of cleaning rod 216, respectively. In order to enhance the ability to reach and irrigate clogging matter residing around the circumference of the lumen, irrigation nozzle 214 may include a plurality of water jets which may direct irrigation fluid in different directions. For example, forward jet 213 spurts irrigation fluid in a straight forward direction and is intended to irrigate clogging matter residing in the lumen ahead of cleaning rod 216. Vortex jet 212 spurts irrigation fluid in a radial direction, which allows reaching and drenching clogging matter which does not reside straight ahead of cleaning rod 216 but rather in a relative sideways position. In addition, vortex jet may create a turbulence within the lumen fluids which assists evacuation of the fluids through endoscope bore 190 and peripheral conduit 110 and out of suction port 112. Operation of cleaning rod 216, irrigation nozzle 214 and camera tube 215 in the absence of an endoscope, i.e., when a sealing plug is installed as shown in FIG. 9D, may have substantially improved results in clearing clogging matter and lumen fluids from the lumen. Absence of an endoscope may allow easier access to fecal debris and other clogging matter, and an unobstructed flow of lumen fluids out through suction port 112. This may lead to a relatively smooth advancement of an endoscope when inserted. In order to further improve evacuation of lumen fluids from the lumen, endoscope bore 190 may include helical grooves, configured to initiate and/or improve vortex flow of lumen fluids. The additional flow directionality allows overcoming of possible hindrances and better access of the lumen fluids to exit through suction port 112.

FIG. 14C is a schematic illustration of adaptor 400 and cleaning rod 216, coupled with endoscope 140. FIG. 14C further emphasizes the peripheral position of cleaning rod 216 in relation to endoscope 140 when endoscope 140 is inserted through endoscope bore 190.

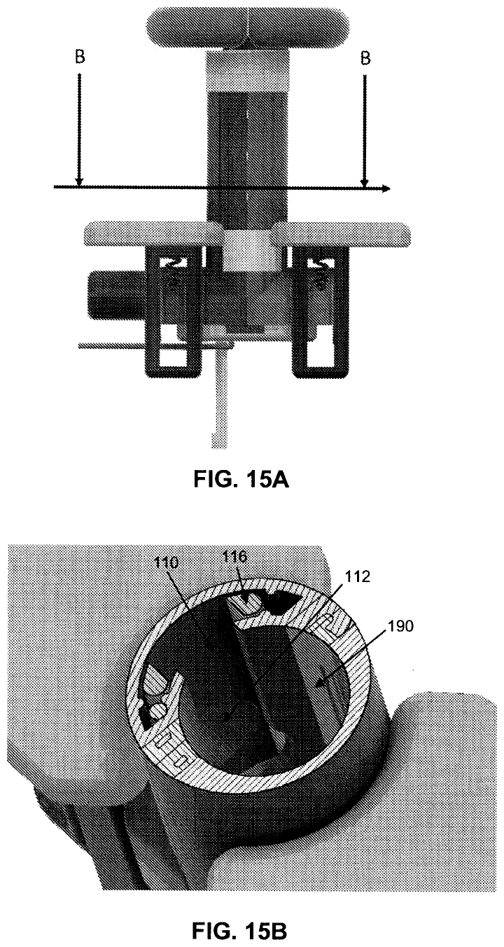

Reference is now also made to FIGS. 15A to 15B which show a cross section of adaptor 100 displaying components of fluid conducting arrangement 109. FIG. 15A is a schematic illustration of adaptor 100. FIG. 15B is a schematic, cross-sectional illustration of adaptor 100 taken along line B-B denoted in FIG. 15A. The cavities of endoscope bore 190 and peripheral conduit 110 and the open pathway between them are shown. Cleaning rod 116 is shown inserted into peripheral conduit 110, leaving an open space between its two arms 116a and 116b and suction port 112 is shown open to peripheral conduit 110, allowing fluid communication between endoscope bore 190, peripheral conduit 110 and suction port 112.

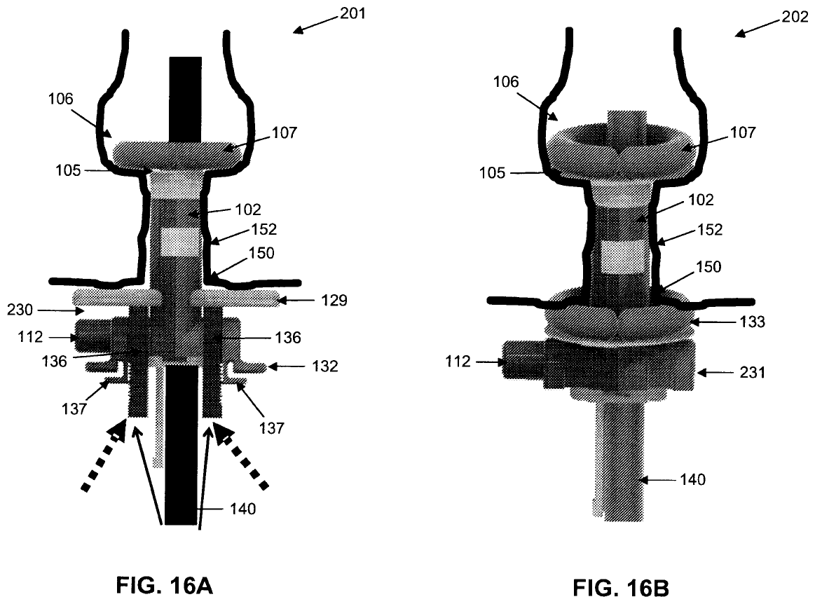

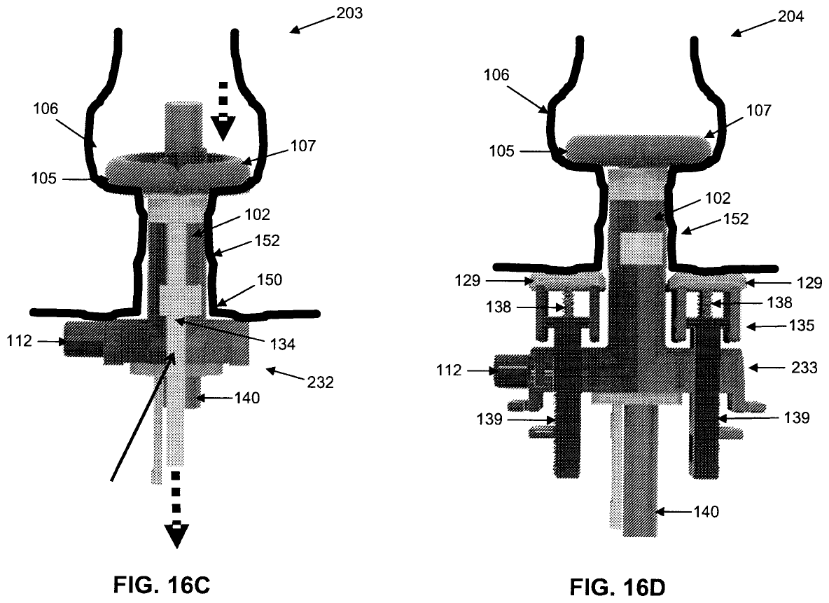

Reference is now also made to FIGS. 16A to 16D which illustrate alternative fixating mechanisms of body recliner 130 of an embodiment of lumen fluid control and endoscope coupling adaptors 201-204, constructed and operative in accordance with the invention. Adaptors 201-204 are shown coupled with endoscope 140 and inserted into proximal portion of the lumen 152. FIG. 16A is a schematic illustration of adaptor 201 where proximal external body-recliner 230 includes cushion 129 coupled with ratchet 132. Ratchet 132 features toothed bar 136 which is movable with respect to snapping tooth-stopper 137, allowing adjusting the longitudinal dimension of recliner 230 for fitting the length of proximal portion of the lumen 152. Following insertion of protruding muzzle 102 into proximal portion of the lumen 152, cushion 129 interfaces with the buttocks of patient's body 170 and is pushed rearwards in relation to the direction of the insertion. Movable toothed bar 136, which is coupled with cushion 129, moves along snapping tooth-stopper 137 until protruding muzzle 102 completes its insertion into the lumen and is kept in place by tooth-stopper 137.

FIG. 16B is a schematic illustration of adaptor 202, where proximal external body-recliner 231 includes external inflatable balloon 133. The degree of inflation of external inflatable balloon 133 is regulated, allowing adjusting the longitudinal dimension of recliner 231 for fitting the length of proximal portion of the lumen 152. Following insertion and final positioning of adaptor 202, balloon 133, which interfaces with the buttocks of patient's body 170, may be further inflated in order to exert a pulling force on adaptor 202 to assist fixation and sealing by clutch 106.

FIG. 16C is a schematic illustration of adaptor 203, where pulling strap 134 fixed to collapsible clutch 106 serves to adjust and fix the longitudinal position of adaptor 203. Pulling strap 134 is fixed to clutch 106, which is mobile along the length of protruding muzzle 102, and extends out of lumen entry 150. On strap 134's externally extending end is a pulling portion, allowing to tighten and adjust clutch 106's longitudinal position along muzzle 102 and in relation to the lumen in order to fixate adaptor 203 and seal the gap between proximal portion of the lumen 152 and muzzle 102. One strap or more may be used.

FIG. 16D is a schematic illustration of adaptor 204, where proximal external body-recliner 233 includes cushion 129 coupled with resilient members connected to a ratchet 135. Resilient members connected to a ratchet 135 includes resilient members 138 and ratchet 139, where ratchet 139 features toothed bar 136 which is movable with respect to snapping tooth-stopper 137. Resilient members connected to a ratchet 135 allows adjusting the longitudinal dimension of recliner 233 for fitting the length of proximal portion of the lumen 152, and further serves to exert a pulling force on adaptor 204 and to assist fixation and sealing by clutch 106. Following insertion of protruding muzzle 102 into proximal portion of the lumen 152, cushion 129 interfaces with the buttocks of patient's body 170 and is pushed rearwards in relation to the direction of the insertion. Movable toothed bar 136 of ratchet 139, which is coupled with resilient members 138 coupled in turn with cushion 129, moves along the snapping tooth-stopper until protruding muzzle 102 completes its insertion into the lumen, and is then kept in place by tooth-stopper 137. The length of adaptor 204 is thus adjusted in accordance with the length of proximal portion of the lumen 152. Pushing rearwards of cushion 129 also presses on resilient members 138, inducing a pushing force of cushion 129 on the buttocks of patient's body 170 and a pulling force on adaptor 204, for assisting fixation of adaptor 204 and sealing of the lumen by clutch 106.

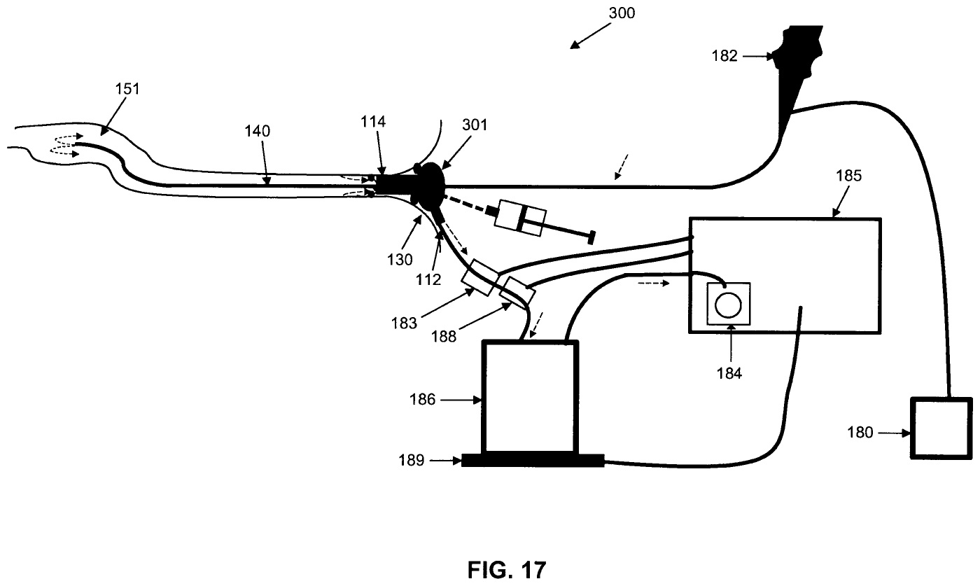

Reference is now also made to FIG. 17 which is a schematic, cross-sectional illustration of an embodiment of a lumen fluid control and endoscope coupling adaptor 301 and external fluid management system 300, constructed and operative in accordance with the invention. System 300 features a lumen fluid suction unit in fluid communication with suction port 112, for evacuation of fluids from the lumen, and an irrigation unit in fluid communication with the lumen, for streaming irrigation fluids into the lumen. One embodiment of a lumen fluid suction unit may be vacuum suction pump 184, and one embodiment of an irrigation unit may be irrigation fluid pump 180. System 300 further features endoscope fluid adaptor 182 which is coupled to endoscope 140, sewage canister 186, and external fluid controller 185. External fluid controller 185 includes and controls the operation of measuring sensors, including vacuum suction pump 184, irrigation fluid pump 180, pressure sensor 183, fluids flow sensor 188, and weighing scale 189. Adaptor 301, which is coupled with endoscope 140, is inserted into proximal portion of the lumen 152. In full operation of the system, water or other cleansing fluid is pumped by fluid pump 180 through endoscope fluid adaptor 182 into endoscope 140 and therein through the length of the endoscope into the lumen, distally irrigating lumen 151. Vacuum suction pump 184 may be positioned upstream of sewage canister 186, i.e., between suction port 112 and sewage canister 186, or positioned downstream of sewage canister 186. Vacuum suction pump 184 is in fluid communication with suction port 112 and with sewage canister 186. When vacuum suction pump 184 is positioned downstream of canister 186, the fluid communication with suction port 112 is through canister 186. Pump 184 applies a suction force on the fluids in the lumen through suction port 112, causing the fluids to drain through peripheral conduit 110 and endoscope bore 190, out of suction port 112 and into sewage canister 186. Vacuum pump 184 may be replaced by any other type of lumen fluid suction unit that is in fluid communication with suction port 112. Sewage canister weighing scale 189, which is connected to controller 185, may be used to measure the amount of fluid in sewage canister 186 and to help estimate the amount of cleansing fluid remaining inside lumen 151 of the patient. Pressure sensor 183 and fluids flow sensor 188 may be used to measure, calculate and/or control the vacuum level at suction port 112 and to measure the fluid flow at suction port 112, respectively. Cleaning rod 116 may be applied as illustrated in FIG. 11 to assist in clearing peripheral conduit 110 and the lumen by breaking up peripheral clogging matter.

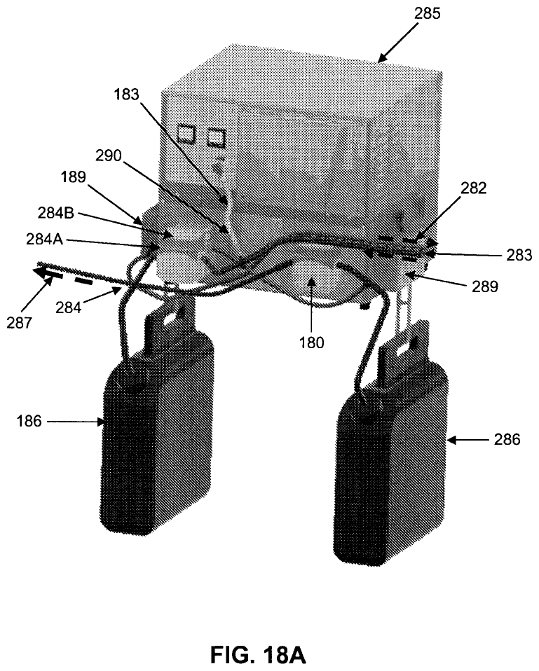

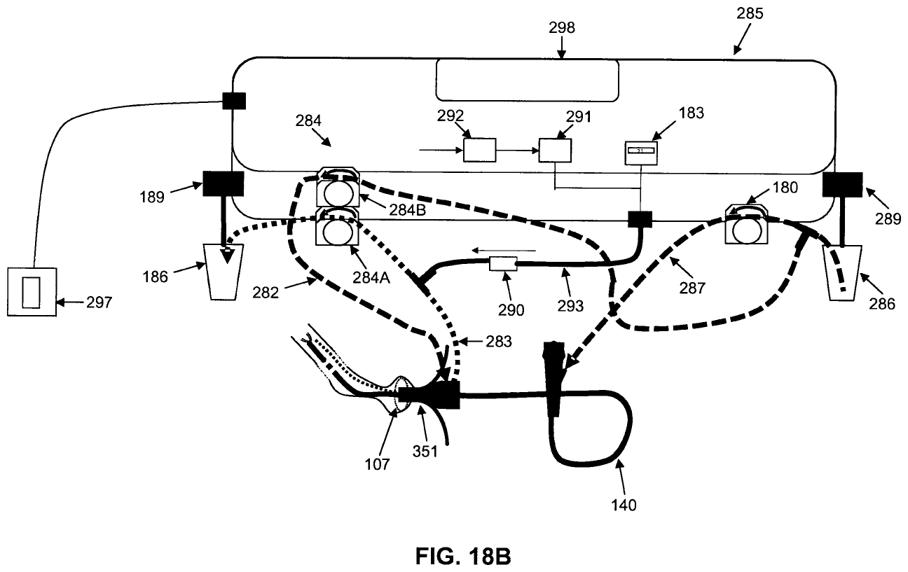

Reference is now also made to FIG. 18A, which is a schematic illustration of a balanced external fluid controller 285 with pumps 180,284 and dual weighing scales 189,289 of an external fluid management system, constructed and operative in accordance with the invention. Reference is also made to FIG. 18B, which is a schematic cross-sectional illustration of an embodiment of a lumen fluid control and endoscope coupling adaptor 351 and an external fluid management system 350, including balanced external fluid controller 285, constructed and operative in accordance with the invention.

For clarification, in FIG. 18B tubes 282, 283, 287 and 293 are all tubes through which fluids flow, where: a dotted line (i.e., tube 283) signifies a tube carrying fluids evacuated from the lumen (also described as “lumen fluids”); a dashed line (i.e., tubes 282, 287) signifies a tube carrying irrigation fluids towards the lumen; and a hollow line (i.e., tube 293) signifies a tube through which air is streamed.

Balanced external fluid controller 285 may include and control measuring sensors, including irrigation canister weighing scale 289 and sewage canister weighing scale 189, which measure the fluid quantity in irrigation canister 286 and sewage canister 186, respectively. Irrigation canister 286 contains a supply of irrigation fluid for pumping into the lumen. Sewage canister 186 collects the lumen fluids which have been evacuated from the lumen. Irrigation canister 286 and sewage canister 186 may be any form of container suited for containing liquids, e.g., a bag. Balanced external fluid controller 285 may include and control the operation of irrigation fluid pump 180, vacuum suction pump 284A, and adaptor irrigation pump 284B. Irrigation fluid pump 180 is connected, via endoscope irrigation tube 287, on one end to irrigation canister 286 and on the other end to endoscope 140, and is configured to pump irrigation fluid out of irrigation canister 286 through endoscope irrigation tube 287 into the proximal end of endoscope 140. Vacuum suction pump 284A is another embodiment of a lumen fluid suction unit, used for evacuation of lumen fluids through suction port 112 of adaptor 351, functioning similarly to vacuum suction pump 184 of FIG. 17. Vacuum suction pump 284A is connected, via fluid evacuation tube 283, on one end to suction port 112 and on the other end to sewage canister 186, and is configured to suction fluids from the lumen through evacuation tube 283 into sewage canister 186. Adaptor irrigation pump 284B is another embodiment of an irrigation unit. Pump 284B is connected, via adaptor irrigation tube 282, on one end to irrigation canister 286 and on the other end to adaptor 351, and is configured to pump irrigation fluid out of canister 286 into the lumen through adaptor irrigation tube 282 and through a component of the adaptor 351, e.g., an irrigation nozzle 214 as shown in FIGS. 14A, 14B. Vacuum suction pump 284A and adaptor irrigation pump 284B may be coupled in a dual head pump 284. Dual head pump 284 may contain a single motor, and pumps 284A, 284B may be positioned or otherwise organized such that powering of the motor causes suction pump 284A to suction fluids out of the lumen and irrigation pump 284B to stream irrigation fluids into the lumen simultaneously. For example, suction pump 284A may be mounted together with adaptor irrigation pump 284B on a common axis. Pumps 284A, 284B may be relatively positioned such that the rotation of the common axis causes suction pump 284A to suction fluids out of the lumen, and causes irrigation pump 284B to stream irrigation fluids into the lumen. Allowing pumps 284A, 284B to be handled by the operation of a common motor simplifies the components and the operation of controller 285. More importantly, the common motor and axis of the two pumps 284A, 284B allows activating vacuum suction pump 284A and adaptor irrigation pump 284B in a coordinated manner, where activating, deactivating, and adjusting the pumping force are fully synchronized. It will be appreciated that a synchronized operation of vacuum suction pump 284A and adaptor irrigation pump 284B may provide a dynamic flow of irrigation fluids into the lumen and lumen fluids out of the lumen, while maintaining a basically constant quantity of fluids within the lumen which may be required. This may be especially useful when sealing plug 413 (as shown in FIG. 9D) is installed at the proximal end of adaptor 351 in place of endoscope 140, and may therefore be particularly advantageous to coordinate the operation of adaptor irrigation pump 284B, which does not depend on the presence of endoscope 140, with vacuum suction pump 284A.

External fluid controller 285 may include and control measuring sensors, including pressure sensor 183 which may be connected to evacuation tube 283 of vacuum suction pump 284A. Pressure sensor 183 is configured to measure the vacuum pressure in evacuation tube 283. The vacuum pressure which vacuum suction pump 284A induces in evacuation tube 283 could lead to the accumulation of a high vacuum pressure within the lumen, which may possibly cause the lumen walls to collapse. In proper function of external fluid management system 350 a high vacuum pressure within the lumen is unlikely to build up, particularly when vacuum suction pump 284A is synchronized with adaptor irrigation pump 284B. However, a technical fault in the system or unmonitored emptying of irrigation canister 286 could bring about a situation where suction pump 284A is applying a vacuum force toward the lumen while there are insufficient lumen fluids to be suctioned by the applied vacuum force. To overcome this issue, controller 285 may operate pressure sensor 183 to continuously measure the pressure levels in evacuation tube 283. When the pressure levels drop below a predefined threshold (i.e., the vacuum level is above a predefined threshold) controller 285 may be configured to activate a countermeasure. For example, controller 285 may include and control air streaming means, including valve 291 and air pump 292, and vacuum suction pump 284A may include air tube 293. Air tube 293 is connected and in fluid communication with evacuation tube 283. One-way check valve 290 may be installed within air tube 293, preferably in proximity to the connection point with evacuation tube 283, and controlled by controller 285. Air pump 292 is in fluid communication with air tube 293 via air valve 291, which is configured to open and allow air to be pumped by air pump 292 into air tube 293 when air pump 292 is activated by controller 285, and to otherwise remain sealed. When the pressure in evacuation tube 283 descends below a predefined threshold, air pump 292 is activated to stream air into evacuation tube 283 through air valve 291, air tube 293 and one-way check valve 290. This immediately raises the air pressure in evacuation tube 283 and halts the accumulation of vacuum force in the lumen, while allowing continued operation of vacuum suction pump 284A. Alternatively, controller 285 may open air valve 291 without operating air pump 292, relying on the low pressure in evacuation tube 283, i.e., the pressure difference between evacuation tube 283 and the atmosphere, to exert a drawing force and draw atmospheric air through air valve 291 and air tube 290 into evacuation tube 283. Pressure adjustment in evacuation tube 283 using air tube 293 is particularly significant when vacuum suction pump 284A and adaptor irrigation pump 284B are coupled in dual head pump 284 with a common motor and axis, as it allows continued activity of adaptor irrigation pump 284B to introduce irrigation fluids into the lumen. One-way check valve, inserted within air tube 293, serves to maintain a one-way flow direction only towards evacuation tube 283, and to prevent contamination from evacuation tube 283 from passing through air tube 290.

As may be seen, irrigation fluids may be introduced into the lumen either through endoscope 140 or through adaptor 301, for example via Irrigation nozzle 214. As explained with reference to FIGS. 9D and 14B, lumen irrigation may be performed through components of the adaptor when an endoscope is not disposed within endoscope bore 190, which may allow improved access to fecal debris and other clogging matter within the lumen.

Irrigation canister 286 may be connected to an irrigation fluid source and constantly refilled, or may not be connected to an irrigation fluid source, containing a specific volume of irrigation fluid at the start of the treatment from which cleansing fluid is pumped into the lumen throughout the treatment as needed. An irrigation canister 286 that is not connected to a fluid source may be advantageous in a system including dual weighing scales 189,289. Constant measuring of the quantity of fluids in both irrigation canister 286 and sewage canister 186 using weighing scales 289 and 189, respectively, may substantially enhance the ability to accurately estimate the volume of fluids that are present in the lumen, by comparing the volume of fluids pumped out of irrigation canister 286 and those accumulated in sewage canister 186. Together with the other sensors, this may improve endoscope handling and maneuvering abilities within the lumen.

External fluid controller 285 may additionally include touch screen control panel 298, which may allow easy controlling of the operation of any measuring sensors, e.g., pressure sensor 183, and any pumps, e.g., vacuum suction pump 284A, which are controlled by controller 285. Each of the measuring sensors and pumps may be individually represented on panel 298, and touching the screen at designated points may be operational to switch on, switch off, raise, lower, or otherwise adjust the operation of any of the measuring sensors or pumps. Touch screen control panel 298 may additionally continuously show the measurements made by the measuring sensors, and controller 285 may be configured to present instructions to the operator on panel 298, which derive from the measurements made by the measuring sensors. Controller 285 may also include foot pedal 297, which may allow the operator to switch on, switch off, raise, lower, or otherwise adjust the operation of any of the measuring sensors or pumps by stepping on foot pedal 297, without the need of using his hands, which may often be otherwise occupied in the course of an endoscopic procedure.

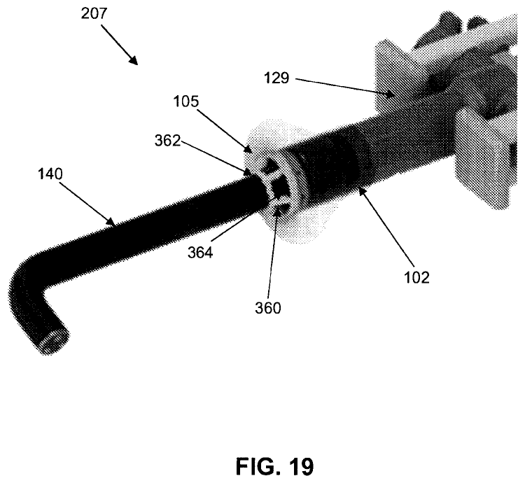

Particularly upon activation of vacuum suction pump 284A for removal of lumen fluids, the applied suction force may produce a pressure on the lumen walls which may cause the lumen walls to cave in towards the distal end of the adaptor (100). Upon collapse, at least a part of the lumen walls may enter a cavity located at the distal end of the adaptor and get caught or pinched therein, where the cavity may include, for example, the gap between endoscope 140 and the inside walls of endoscope bore 190, or the distal opening of peripheral conduit 110. To prevent, or minimize, constriction of the lumen wall, a lumen protecting element may be added to the distal end of the adaptor. Reference is now made to FIG. 19, which is a schematic illustration of a further embodiment of a lumen fluid control and endoscope coupling adaptor 207 constructed and operative in accordance with the invention. Adaptor 207 features a lumen protective crown 360 installed at a distal end of protruding muzzle 102, where protective crown 360 is a non-limiting exemplary embodiment of a lumen protecting element. Lumen protective crown 360 is ring-shaped, the interior portion of the ring proximately encircling endoscope 140 at a region of endoscope 140 which is distal to adaptor 207, and the outer perimeter of the ring being coupled with the distal end of the walls of protruding muzzle 102. One or more apertures 364 are intermittently spaced about the perimeter of crown 360, spanning between endoscope 140 and protruding muzzle 102. Protective crown 360 may be ring shaped, or may have any other relevant shape, e.g., a series of spindle-like legs dispersed along the circumference of protruding muzzle 102 and extending towards endoscope 140. Protective crown 360 may completely or partially encircle the distal portion of adaptor 207. The partial region of adaptor 207 which protective crown 360 may span may be determined according to the region of the adaptor in which there is a higher risk of the lumen wall collapsing and becoming caught/wedged within peripheral conduit 110 or endoscope bore 190. Lumen protective crown 360 may be coupled with protruding muzzle 102, clutch 106, the lumen seal, or any other component of adaptor 207 which extends into the lumen.

Protective crown 360 may surround endoscope 140 at various degrees of proximity, e.g., tightly or loosely clasping endoscope 140. As well as the general aim of protective crown 360 to hinder entry of the lumen wall into cavities at the distal end of the adaptor, the interior portion of crown 360 may also function to separate, or peel, the lumen wall off endoscope 140 when endoscope 140 is retracted. Usually, protective crown 360 surrounds endoscope 140 at a relatively high proximity, so as to limit penetration of the lumen wall into the gap between endoscope 140 and crown 360, and to effectively separate the lumen wall from endoscope 140 when retracting, yet loosely enough so as not to compromise the maneuverability of endoscope 140 within the lumen (i.e., turning, advancing and/or retracting). Optionally, protective crown 360 may be adjustably contractible, such that when endoscope 140 is stationary crown 360 can be constricted to tightly grasp endoscope 140, and when endoscope 140 is intended to be maneuvered within the lumen the interior portion of crown 360 can be retracted (minimally) therefrom.

The size of the apertures 364 is a function of conflicting objectives: on the one hand, the objective of allowing lumen fluids (including fecal debris) to be evacuated from the lumen through endoscope bore 190 and peripheral conduit 110, for which purpose apertures 364 should be as large as possible; and on the other hand, the objective of hindering entry of the lumen wall into peripheral conduit 110 etc., for which purpose apertures 364 should be as small as possible. Even when apertures 364 are narrow enough to prevent pinching, the lumen wall may be sucked toward the openings in the distal end of adaptor 207 (by the suction applied by vacuum suction pump 284A), and may partially cave in such that the lumen wall covers and substantially blocks at least one of apertures 364. This may severely hinder the evacuation of lumen fluids in particular, and the lumen clearing operation in general. Blockage of apertures 364 (or of endoscope bore 190/peripheral conduit 110, when a protective crown 360 is not installed, or is not effective in keeping-out the lumen wall) may be sensed by any one of various sensors, such as a pressure sensor, a flow sensor, and the like. Upon sensing a change in pressure level, flow level, etc., which is indicative of a blockage at the distal end of protruding muzzle 102 (e.g., blockage of apertures 364), an indication may be provided by a controller (285) to an operator, according to which indication the operator may activate, deactivate, and/or adjust the settings of at least one of the pumps or other components of the fluid management system. For example, the operator may lower the suction force applied by vacuum suction pump 284 toward the lumen, may halt the operation of vacuum suction pump 284 altogether, or may reverse the direction of the pump, such that vacuum suction pump 284 streams air toward the lumen instead of suctioning air (and liquids) from the lumen.

Protective crown 360 may be made up of (at least) two connectible parts, joined on a common axis at one end and splitable at the opposite end, in a mechanism and design similar to, for example, fastenable shells 420, 421 and splitable narrowing grommet 412 (FIGS. 8A, 8B, and 9C). When protective crown 360 is in this form it may be installed at the distal end of fastenable shells 420, 421, such that when the fastenable shells are shut about endoscope 140, protective crown is also fastened about a portion of endoscope 140 which extends beyond the distal end of the protruding muzzle.

Alternatively or additionally, the lumen protective element may extend substantially vertically from the distal end of adaptor 207 along the inner wall of the lumen, and optionally may slant toward the inner wall of the lumen (i.e., slanting radially outwards from endoscope 140), so as to support the lumen wall and repress its collapse onto endoscope 140 and into the distal end of the adaptor 207. The lumen protecting element may include other means for supporting the lumen wall when suction is applied, such as deployable arms which may be coupled with the external wall of endoscope 140, optionally at several points along the length of endoscope 140, and can be extended toward the lumen wall according to need; vertically extendable/inflatable cushions, which may be coupled to the distal portion of muzzle 102 and can be extended vertically along the lumen to push out vertically and support the lumen wall; and the like.

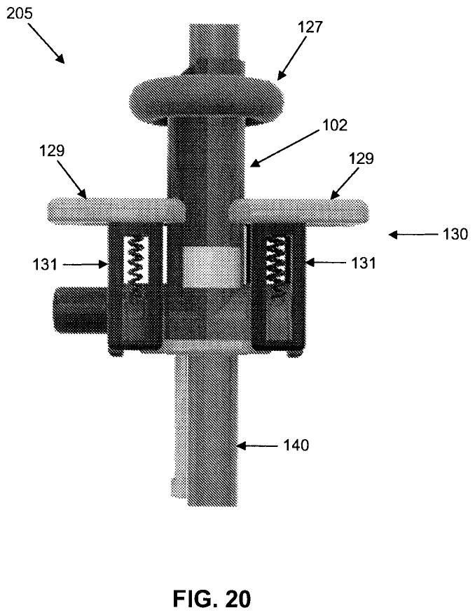

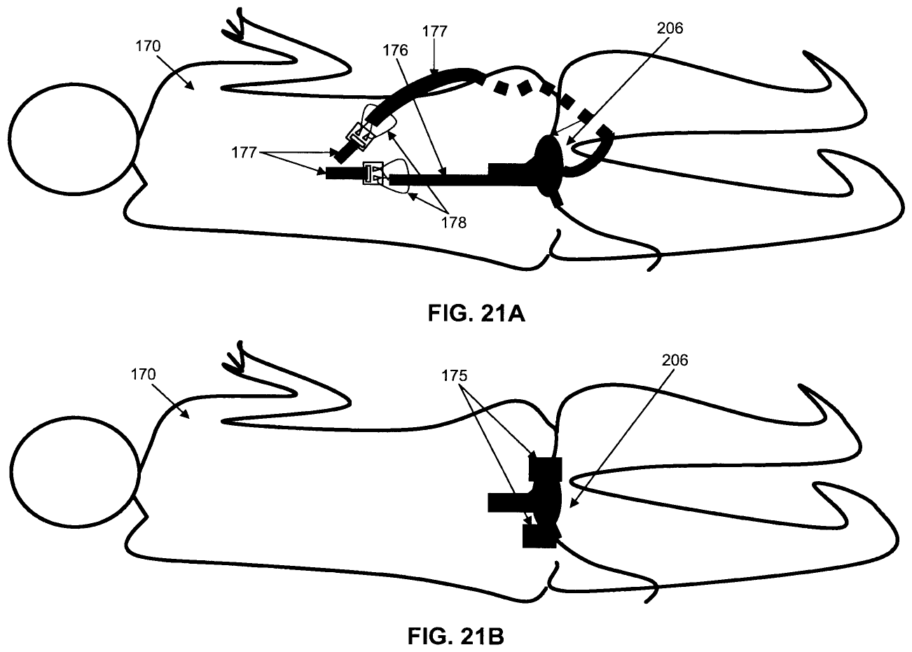

Reference is now also made to FIG. 20 which is a schematic side view illustration of an embodiment of a lumen fluid control and endoscope coupling adaptor 205 constructed and operative in accordance with the invention, featuring distal clutch 106, including inflatable torus balloon 127. Balloon 127 is operational when inflated to prevent backflow of the lumen fluids by sealing the gap between protruding muzzle 102 and the lumen, when adaptor 205 is inserted within the lumen. Reference is now also made to FIGS. 21A to 21B which show embodiments of a lumen fluid control and endoscope coupling adaptor 206, constructed and operative in accordance with the invention shown inserted into proximal portion of the lumen 152, and fixated using straps 176, tapes 175,177 and pulling wires 178 attached to patient's body 170. FIG. 21A is a schematic side view illustration of adaptor 206 attached to patient's body 170 using straps 176 wires 178 and tapes 177. Straps 176 are connected to adaptor 206, and on them are fitted pulling wires 178 for tightening and adjusting straps 176, where wires 178 are brought around the patient's waist, and attached to the patient's back using tapes 177.

FIG. 21B is a schematic side view illustration of adaptor 206 attached to patient's body 170 with tape 175. Tape 175 is connected at one end to adaptor 206 and at the other end to the patient's buttocks, serving to further fixate adaptor 206 in place.

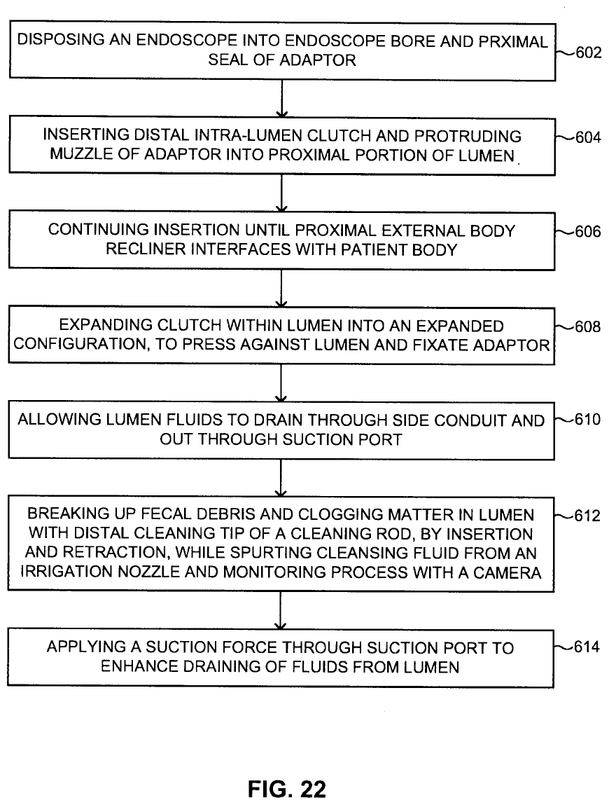

Reference is now made to FIG. 22 which is block diagram of a method 600 for endoscope coupling and fluid conducting, operative in accordance with an embodiment of the present invention.

Step 602 includes disposing an endoscope into an adaptor, the adaptor including a longitudinally extending endoscope bore bored through the adaptor, and a proximal seal configured to surround an endoscope inserted in the bore, effecting blocking of fluid leakage from the proximal end of the adaptor. With reference to FIGS. 1 to 5, endoscope 140 is disposed into adaptor 100, adaptor 100 including longitudinally extending endoscope bore 190 bored through adaptor 100 and proximal seal 111 configured to surround endoscope 140 inserted in bore 190, effecting blocking of fluid leakage from the proximal end of adaptor 100.

Step 604 includes inserting the distal end of the adaptor into a proximal portion of the patient's lumen, the adaptor including a distal intra-lumen collapsible clutch, an interim protruding muzzle and a proximal external body recliner. With reference to FIGS. 6 and 10A to 100, distal end of adaptor 100 is inserted into proximal portion of the patient's lumen 152, adaptor 100 including distal intra-lumen collapsible clutch 106, interim protruding muzzle 102 and proximal external body-recliner 130.

Step 606 includes continued inserting of the adaptor until the external body recliner interfaces the patient's body, where a distal lumen seal is operative to block leakage from a gap between the distal end of the protruding muzzle and the lumen. With reference to FIGS. 6, 10A to 100 and 17, adaptor 100 (or 301) is continuously inserted until external body-recliner 130 interfaces patient's body 170, where distal lumen seal 114 is operative to block leakage from a gap between the distal end of protruding muzzle 102 and the lumen.

Step 608 includes expanding the distal collapsible clutch within the proximal portion of the lumen into an expanded configuration which presses against the lumen to fixate the adaptor to the body of the patient. With reference to FIGS. 10A to 100, collapsible clutch 106 expands within proximal portion of the lumen 152 into an expanded configuration, which presses against the lumen to fixate adaptor 100 to the patient's body 170.

Step 610 includes allowing lumen fluids to drain through a longitudinally extending peripheral conduit bored along the adaptor through the muzzle, and to be released through a fluid suction port which is in fluid communication with the peripheral conduit, where the proximal seal blocks the fluids from leaking from the proximal end of the adaptor. With reference to FIGS. 3 and 15A to 15B, lumen fluids are allowed to drain through longitudinally extending peripheral conduit 110 bored along adaptor 100 through muzzle 102, and to be released through fluid suction port 112 which is in fluid communication with peripheral conduit 110, where proximal seal 111 blocks the fluids from leaking from the proximal end of adaptor 100.

Step 612 includes physically breaking up and grinding of fecal debris and other clogging matter using a cleaning rod featuring a distal cleaning tip, where the rod is sealingly inserted through the proximal seal and the peripheral conduit, and the grinding is accomplished by inserting and retracting the rod ahead of or peripherally of the endoscope. Cleansing fluid is spurted into the lumen from an irrigation nozzle, and a camera tube allows viewing and monitoring of the cleaning process, where the irrigation nozzle and the camera tube are extending through one or more hollow arms of the cleaning rod. With reference to FIGS. 13A to 13C and 14A to 14C, cleaning rod 216 featuring a distal cleaning tip, physically breaks up and grinds fecal debris and other clogging matter within the lumen, where rod 216 is sealingly inserted through proximal seal 111 and peripheral conduit 110. The grinding is accomplished by inserting and retracting rod 216 ahead of or peripherally of endoscope 140. Irrigation nozzle 214 and camera tube 215 extend into the lumen through hollow arms 216a,216b of cleaning rod 216, for spurting cleansing fluid and viewing and monitoring the cleaning process, respectively.

Step 614 includes applying a suction force to the lumen fluids through the suction port in order to enhance draining of fluids from the lumen. A lumen protecting element may be provided at the distal end of the adaptor, to hinder a lumen wall from caving inwards due to the suction pressure, and getting caught or pinched within a cavity located at the distal end of the adaptor. With reference to FIGS. 17 and 19, a suction force is applied to the lumen fluids through suction port 112 in order to enhance draining of fluids from the lumen. Lumen protective crown 360 may be installed at a distal end of adaptor 207 (e.g., between the distal end of protruding muzzle 102 and endoscope 140), to hinder a lumen wall from entering peripheral conduit 110, endoscope bore 190, or any other cavity at the distal end of the adaptor, particularly as a result of suction pressure.

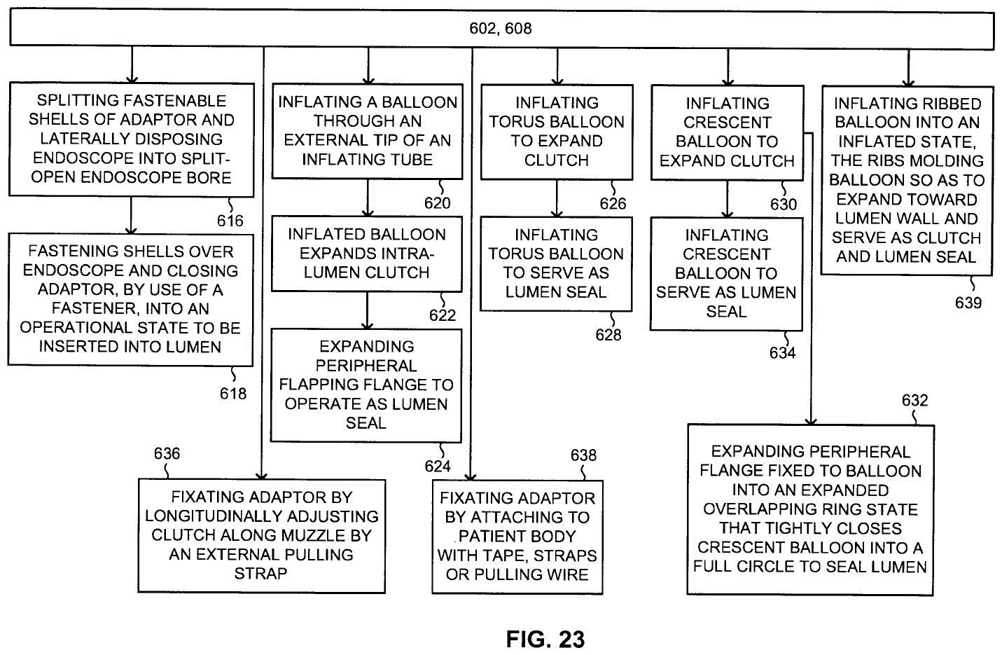

Method 600 may include further optional or preferable procedures and features outlined below. Reference is now made to FIG. 23 which is a block diagram of sub-routines or sub-procedures of steps 602 and 608 of the embodiment of FIG. 22.

Step 616 includes lateral laying of the endoscope in the endoscope bore and the proximal seal, the adaptor including fastenable shells allowing splitting the endoscope bore, the proximal seal, and the clutch into an open state. With reference to FIGS. 3 and 7A to 7C, endoscope 140 is laterally laid in endoscope bore 190 and proximal seal 111, adaptor 100 including fastenable shells 120,121 allowing splitting endoscope bore 190, proximal seal 111, and clutch 106 into an open state.

Step 618 includes fastening of the fastenable shells over the disposed endoscope into a closed state using a fastener, while facilitating insertion and retraction of the endoscope. With reference to FIGS. 7A to 7C and 8A to 8B, fastenable shells 420,421 are fastened over disposed endoscope 140 into a closed state by use of fastener 430, while facilitating insertion and retraction of endoscope 140.

Step 620 includes inflating a balloon through an inflating tube in fluid communication with the balloon, where the tip of which tube remains external to the lumen following insertion, for allowing external access for inflating and deflating the balloon.

Step 622 includes fixating by inflating an internal inflatable balloon connected to the collapsible intra-lumen clutch, where the inflating of the balloon expands the clutch into its expanded state.

Step 624 includes lumen sealing by expanding a distal flapping peripheral flange operational as the lumen seal. When in an expanded state, the flange prevents backflow of the lumen fluids by sealing the gap between the protruding muzzle and the lumen when the adaptor is inserted within the lumen. With reference to FIGS. 11A to 110, distal flapping peripheral flange 105 is operational as the lumen seal. When in an expanded state, flange 105 prevents backflow of the lumen fluids by sealing the gap between protruding muzzle 102 and the lumen, when adaptor 100 is inserted within the lumen.

Step 626 includes inflating a torus balloon fixed to the collapsible distal intra-lumen clutch, for expanding the clutch into its expanded state.

Step 628 includes lumen sealing by inflating of a distal inflatable torus balloon, operational when inflated to prevent backflow of the lumen fluids by sealing the gap between the protruding muzzle and the lumen when the adaptor is inserted within the lumen. With reference to FIG. 20, distal inflatable torus balloon 127 is inflated, operational when inflated to prevent backflow of the lumen fluids by sealing the gap between protruding muzzle 102 and the lumen when adaptor 205 is inserted within the lumen.

Step 630 includes inflating an inflatable crescent balloon fixed to the clutch, for expanding the clutch into its expanded state. With reference to FIGS. 10A to 10C, inflatable crescent balloon 107 fixed to clutch 106 is inflated, for expanding clutch 106 into its expanded state.

Step 632 includes expanding a distal flapping peripheral flange, surrounding the distal end of the muzzle in a more than full circle (e.g., overlappingly wound about a circle at 360°+40°) and coupled to the crescent balloon. Inflating of the crescent balloon expands the peripheral flange into an expanded state, and the consequent overlapping of the flange tightly closes the crescent balloon into a full ring shape (joining the two ends of the crescent). With reference to FIGS. 11A to 110, distal flapping peripheral flange 105 is expanded, surrounding the distal end of protruding muzzle 102 in a more than full circle (e.g., 360°+40°) and coupled to crescent balloon 107. Inflating of crescent balloon 107 expands peripheral flange 105 into its expanded state, and the consequent overlapping of flange 105 tightly closes crescent balloon 107 into a full ring shape (joining the two ends of the crescent).

Step 634 includes lumen sealing by inflating a distal inflatable crescent balloon, operational when inflated for preventing backflow of the lumen fluids by sealing the gap between the muzzle and the lumen when inserted within the lumen. With reference to FIGS. 10A to 10C, distal inflatable crescent balloon 107 is inflated, for preventing backflow of the lumen fluids by sealing the gap between muzzle 102 and the lumen when inserted within the lumen.

Step 636 includes fixating the adaptor by longitudinally adjusting the position of the intra-lumen clutch along the protruding muzzle in relation with the length of the proximal portion of the lumen. The adjusting includes a pulling strap fixed to the clutch, where the clutch is mobile along the length of the muzzle. The strap includes a proximal pulling portion which extends externally of adaptor inserted in the lumen, and this pulling portion allows adjusting of the longitudinal position of the clutch, along the muzzle and in relation to the lumen. With reference to FIG. 16C, longitudinally adjusting the position of intra-lumen clutch 106 along protruding muzzle 102 and in relation with the length of proximal portion of the lumen 152 fixates adaptor 100. The adjusting includes pulling strap 134 fixed to clutch 106, where clutch 106 is mobile along the length of muzzle 102. Strap 134 includes a proximal pulling portion which extends externally of adaptor 203 inserted in the lumen, and this pulling portion allows adjusting of the longitudinal position of clutch 106, along muzzle 102 and in relation to the lumen.

Step 638 includes fixating the adaptor by externally attaching the adaptor to the patient's body using straps, pulling wires and/or tapes. With reference to FIGS. 20A to 20B, adaptor 206 is fixated by externally attaching adaptor 100 to patient's body 170 using straps 176, pulling wires 178 and/or tapes 175,177.

Step 639 includes fixating the adaptor and lumen sealing by inflating a distal ribbed balloon, including semi-rigid ribs attached to the balloon and configured for molding the expansion of the ribbed balloon. When the ribbed balloon is inflated the semi-rigid ribs direct its expansion such that the ribbed balloon expands towards the lumen wall and does not expand towards the endoscope bore, for firmly engaging the lumen walls and sealing the gap between the muzzle and the lumen. With reference to FIGS. 12A to 12B, ribbed balloon 227 positioned at the distal end of a lumen fluid conducting and endoscope coupling adaptor includes semi-rigid ribs 226. Upon inflation of ribbed balloon 227, ribs 226 mould and direct the expansion of ribbed balloon 227 towards the lumen wall and not towards endoscope bore 190. Expansion of ribbed balloon 227 seals the gap between distal protruding muzzle 102 and the lumen wall, and firmly engages the lumen wall to fixate the adaptor.

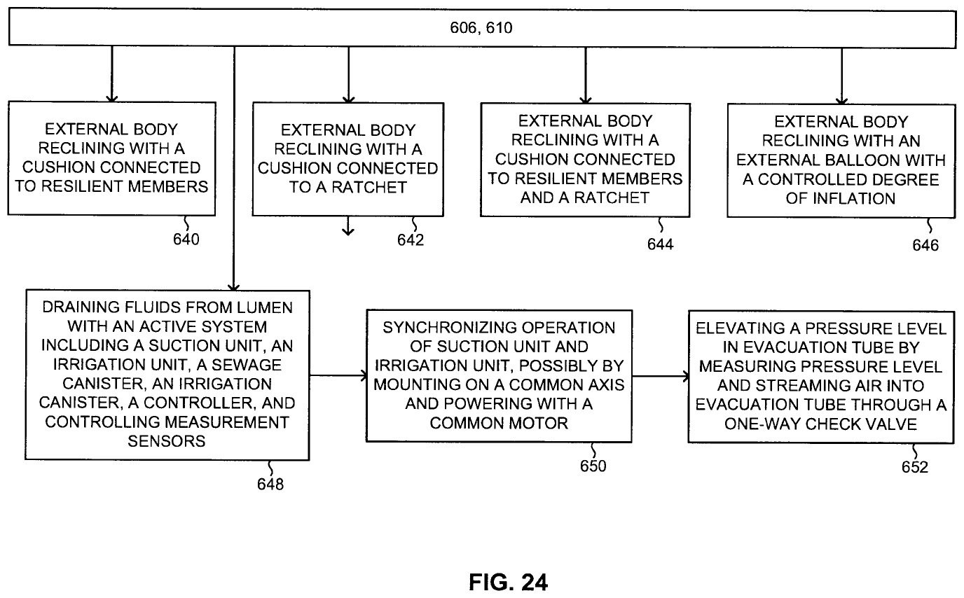

Reference is now made to FIG. 24 which is a block diagram of sub-routines or sub-procedures of steps 606 and 610 of the embodiment of FIG. 22.

Step 640 includes external body reclining by leaning a proximal external cushion fixed to resilient members upon the patient's body, allowing adjusting the longitudinal dimension of the proximal external body-recliner of the adaptor for fitting the length of the patient's proximal portion of the lumen. When the insertion of the adaptor is complete, the resilient members also assist the fixation of the adaptor by exerting a pushing force on the patient's body at the point of interface, and a pulling force on the adaptor, holding the adaptor firmly in place. With reference to FIGS. 10A to 100, proximal external body-recliner 130 including proximal external cushion 129 fixed to resilient members 131, leans upon patient's body 170, resilient members 131 allowing adjusting the longitudinal dimension of proximal external body-recliner 130 for fitting the length of the patient's proximal portion of the lumen 152. When the insertion of adaptor 100 is complete, resilient members 131 also assist the fixation of adaptor 100 by exerting a pushing force on patient's body 170 at the point of interface, and a pulling force on adaptor 100, holding adaptor 100 firmly in place.

Step 642 includes external body reclining by leaning a proximal external cushion fixed to a ratchet upon the patient's body, the ratchet featuring a toothed bar which is movable with respect to a snapping tooth-stopper. This allows adjusting the longitudinal dimension of the proximal external body-recliner for fitting the length of the patient's proximal portion of the lumen. With reference to FIG. 16A, proximal external body-recliner 230 including proximal external cushion 129 fixed to ratchet 132, leans upon patient's body 170. Ratchet 132 features toothed bar 136 which is movable with respect to snapping tooth-stopper 137, allowing adjusting the longitudinal dimension of body-recliner 230 for fitting the length of the patient's proximal portion of the lumen 152.

Step 644 includes external body reclining by leaning a proximal external cushion fixed to resilient members connected to a ratchet upon the patient's body, the ratchet featuring a toothed bar which is movable with respect to a snapping tooth-stopper. This allows adjusting the longitudinal dimension of the proximal external body-recliner for fitting the length of the patient's proximal portion of the lumen. When the insertion of the adaptor is complete, the resilient members also assist the fixation of the adaptor by exerting a pushing force on the patient's body at the point of interface, and a pulling force on the adaptor, holding the adaptor firmly in place. With reference to FIG. 16D, proximal external body-recliner 233 including proximal external cushion 129 fixed to resilient members connected to a ratchet 135, leans upon patient's body 170. Resilient members connected to a ratchet 135 includes resilient members 138 and ratchet 139. Ratchet 139 features toothed bar 136 which is movable with respect to snapping tooth-stopper 137, allowing adjusting the longitudinal dimension of body-recliner 233 for fitting the length of the patient's proximal portion of the lumen 152. When the insertion of adaptor 204 is complete, resilient members 138 also assist the fixation of adaptor 204 by exerting a pushing force on patient's body 170 at the point of interface, and a pulling force on adaptor 204, holding adaptor 204 firmly in place.

Step 646 includes external body reclining by leaning a proximal external inflatable balloon with a regulated degree of inflation upon the patient's body. The flexibility of the balloon's volume allows adjusting the longitudinal dimension of the proximal external body-recliner for fitting the length of the patient's proximal portion of the lumen. When the insertion of the adaptor is complete, the balloon may be further inflated in order to exert a pushing force on the patient's body at the point of interface, and a pulling force on the adaptor, holding the adaptor firmly in place. With reference to FIG. 16B, proximal external body-recliner 231 including proximal external inflatable balloon 133 with a regulated degree of inflation, leans upon patient's body 170. The flexibility of balloon 133's volume allows adjusting the longitudinal dimension of body-recliner 231 for fitting the length of the patient's proximal portion of the lumen 152. When the insertion of adaptor 202 is complete, balloon 133 may be further inflated in order to exert a pushing force on patient's body 170 at the point of interface, and a pulling force on adaptor 202, holding adaptor 202 firmly in place.

Step 648 includes actively suctioning of the lumen fluids through the suction port with a lumen fluid suction unit which applies a suction force to the lumen fluids through the suction port for evacuation of lumen fluids from the lumen, and which may be part of an external fluid management system. The operation of the system may include irrigating the lumen with irrigation fluids by an irrigation unit. The irrigation may include streaming irrigation fluids from an irrigation canister, and collecting the evacuated lumen fluids in a sewage canister. The suctioning from the lumen and the irrigating of the lumen may be controlled by an external fluid controller, which may further include and control measuring sensors for measuring, calculating and/or controlling (1) the fluid flow in the system, particularly at the suction port, (2) the vacuum level at the suction port and/or in an evacuation tube, and (3) the quantity of fluids inside the sewage canister and the irrigation canister. This measuring and calculating may include estimating the amount of cleansing fluid remaining inside the lumen of the patient. The estimating may include measuring the quantity of fluids inside the sewage canister and the irrigation canister by weighing the sewage canister and the irrigation canister. With reference to FIGS. 18A and 18B, lumen fluids are actively drained through suction port 112 by a lumen fluid suction unit, an embodiment of which may be vacuum suction pump 284A, which may be part of external fluid management system 350. An irrigation unit, an embodiment of which may be irrigation pump 284B, streams irrigation fluids into the lumen from irrigation canister 286, and lumen fluids may be collected in sewage canister 186. External fluid controller 285 may control vacuum suction pump 284A and irrigation pump 284B, and further include and control measuring sensors for measuring, calculating and/or controlling: (1) the fluid flow at suction port 112 and/or in tubes 282, 283, 287, and 293, for example with fluids flow sensor 188; (2) the vacuum level at suction port 112 and/or in evacuation tube 283, with pressure sensor 183; and (3) the quantity of fluids inside sewage canister 186 and irrigation canister 286, with weighing scales 189 and 289, respectively. This allows estimating the amount of cleansing fluid remaining inside lumen 151 of the patient.

Step 650 includes synchronizing the operation of the lumen fluid suction unit, in suctioning fluids from the lumen, and the irrigation unit, in streaming irrigation fluids into the lumen, to drain and irrigate the same amount of fluids. The synchronizing may include mounting the lumen fluid suction unit and the irrigation unit onto a common axis and powering them with a common motor of a dual head pump. With reference to FIGS. 18A and 18B, vacuum suction pump 284A, which is an embodiment of a lumen fluid suction unit, and irrigation pump 284B, which is an embodiment of an irrigation unit, are mounted on a common axis and powered by a common motor of dual head pump 284, allowing synchronized operation of pumps 284A, 284B.

Step 652 includes elevating the pressure in the evacuation tube, which communicates the lumen fluid suction unit with the lumen through the suction port, to an elevated pressure above a predefined threshold, without halting the operation of said lumen fluid suction unit. This is accomplished by measuring a pressure level in the evacuation tube and streaming air into the evacuation tube when the pressure level drops beneath the predefined threshold, until the pressure is elevated above the predefined threshold. Preventing contamination from the evacuation tube, e.g., lumen fluids, from flowing through the air tube may be achieved by a one-way check valve which may be disposed into the air tube. With reference to FIG. 18B, pressure sensor 183, of the measuring sensors of external fluid controller 285, measures the pressure in evacuation tube 283, and when the pressure drops beneath a predefined level, controller 285 streams air from air pump 292 through air valve 291 and air tube 290 into evacuation tube 283, to elevate the pressure level in evacuation tube 283 to be above the predefined threshold, without halting the operation of vacuum suction pump 284A. One-way check valve 293 is disposed into air tube 290 to prevent contamination from evacuation tube 283 from passing through air tube 290.

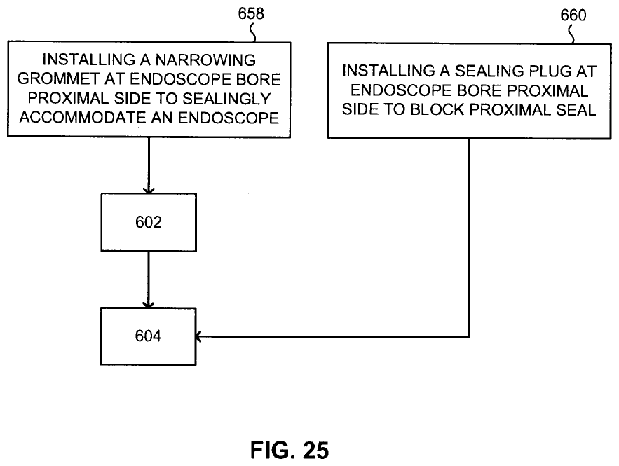

Reference is now made to FIG. 25 which is a block diagram of sub-routines or sub-procedures of steps 602 and/or 604 of the method of FIG. 22.

Step 658 includes installing a narrowing grommet at the proximal end of the endoscope bore, prior to disposing an endoscope into the endoscope bore of an adaptor as in step 602, where the narrowing grommet has an inner circumference suitable to sealingly accommodate an endoscope with a diameter smaller than the diameter of the proximal seal. The narrowing grommet may be installed within the proximal seal or may be proximally attached to the proximal seal, for preventing lumen fluids from leaking out of the proximal side of the endoscope bore. With reference to FIGS. 9A to 9C, narrowing grommet 411 is installed at the proximal end of endoscope bore 190 of adaptor 401. It may be installed within proximal opening 111a of proximal seal 111 or proximally attached to proximal seal 111, prior to insertion of endoscope 440. Narrowing grommet 411 has an inner circumference 411b which is suitable to sealingly accommodate endoscope 440, to prevent lumen fluids from leaking out of the proximal side of endoscope bore 190.

Step 660 includes installing a sealing plug at the proximal end of the endoscope bore when an endoscope is not disposed therein, prior to inserting the distal end of the adaptor into a proximal portion of the patient's lumen, as in step 604. The sealing plug is configured to block the proximal opening of the proximal seal when an endoscope is not disposed within the proximal seal and the endoscope bore, for preventing lumen fluids from leaking out of the proximal end of the adaptor upon insertion into the lumen and conducting the fluids to exit through the suction port. With reference to FIG. 9D, sealing plug 413 is installed at the proximal end of endoscope bore 190 of adaptor 401, prior to insertion of the distal end of adaptor 401 into the patient's proximal portion of the lumen 152. Sealing plug 413 is configured to block proximal opening 111a of proximal seal 111 when an endoscope is not disposed within the proximal opening 111a and endoscope bore 190, in order to prevent leakage of lumen fluids out of the proximal end of adaptor 401 when inserted into the lumen and to conduct the fluids to exit through suction port 112.

Although preferred embodiments are described hereinabove with reference to an endoscope adaptor fixated to the anus and concerting fluid control and suction from the colon, it will be understood that the novel principles of the present invention may be used to fixate instruments and suction/drain fluids from other body cavities, such as, the throat or lungs, and may also be used in lumens and other regions for non-medical applications, as well.

It will thus be appreciated that the preferred embodiments are cited herein by way of example, and the full scope of the invention is limited only by the claims. While certain embodiments of the disclosed subject matter have been described, so as to enable one of skill in the art to practice the present invention, the preceding description is intended to be exemplary only. It should not be used to limit the scope of the disclosed subject matter, which should be determined by reference to the following claims.

1. Lumen fluid conducting and endoscope coupling adaptor for insertion through a lumen entry into a proximal portion of the lumen of a patient and allowing insertion of an endoscope from a proximal end of said adaptor external to the body of the patient to a distal end of said adaptor internally disposed in the lumen, comprising:
a) a proximal external body-recliner, sized to preclude insertion of the recliner into the lumen;
b) an interim protruding muzzle coupled to said body-recliner, sized to allow penetration of said muzzle through said lumen entry into said proximal portion of the lumen;
c) a distal collapsible intra-lumen clutch disposed at the distal end of said protruding muzzle, configured to assume a folded narrow state allowing insertion and retraction through said lumen entry and proximal portion of the lumen, and to assume an expanded state in the lumen, distally of said proximal portion, for fixating said adaptor;
d) a longitudinally extending endoscope bore bored along said adaptor through said muzzle to allow insertion and retraction of the endoscope through said bore into the lumen; and
e) a lumen fluids conducting arrangement, comprising:
i. a longitudinally extending peripheral conduit bored along said adaptor through said muzzle, in fluid communication with said endoscope bore;
ii. a fluid suction port in fluid communication with said peripheral conduit for releasing lumen fluids entering said peripheral conduit from the lumen, said lumen fluids comprising liquids or liquids combined with solids;
iii. an irrigation nozzle, configured to spurt cleansing fluid through said adaptor;
iv. a proximal seal for preventing outward leakage of said lumen fluids, disposed proximally of said adaptor, configured to seal said peripheral conduit and around said endoscope when inserted in said endoscope bore; and
v. a lumen seal for preventing backflow of said lumen fluids by sealing a gap between said protruding muzzle and the lumen when said protruding muzzle is inserted within the lumen.

2. The adaptor according to claim 1, wherein said collapsible intra-lumen clutch comprises at least one selected from the group consisting of:
an inflatable balloon fixed to said clutch, wherein inflating of said balloon expands said clutch into said expanded state;
a flapping peripheral flange which comprises said lumen seal, wherein said flange is operational when in said expanded state to prevent backflow of said lumen fluids by sealing the gap between said muzzle and the lumen, when said adaptor is inserted within the lumen;
an inflatable crescent balloon fixed to said clutch, such that inflating of said crescent balloon expands said clutch into said expanded state; and a flapping peripheral flange, surrounding the distal end of said muzzle in a curl that overlaps a full circle and fixed to said crescent balloon, such that inflating of said crescent balloon expands said peripheral flange into an expanded state, and wherein overlapping of said peripheral flange tightly closes said crescent balloon into a full ring shape; and
an inflatable balloon which is connected to an inflating tube, wherein the tip of which tube remains external to the lumen following insertion of said protruding muzzle, for allowing external control of inflating and deflating said inflatable balloon.

3. The adaptor according to claim 1, wherein said lumen seal comprises at least one selected from the group consisting of:
an inflatable torus balloon, fixed to said clutch, wherein said torus balloon is operational when inflated to expand said clutch into said expanded state and to prevent backflow of said lumen fluids by sealing the gap between said protruding muzzle and the lumen, when said adaptor is inserted within the lumen; and
an inflatable crescent balloon, operational when inflated to prevent backflow of said lumen fluids by sealing the gap between said muzzle and the lumen when inserted within the lumen.

4. The adaptor according to claim 1, wherein said adaptor can be split into at least two fastenable shells, allowing splitting said endoscope bore and said proximal seal into an open state for facilitating disposition of said endoscope in said endoscope bore and said proximal seal, by allowing lateral laying of said endoscope therein before fastening of said fastenable shells into a closed state and facilitating insertion and retraction of said endoscope.

5. The adaptor according to claim 1, comprising a cleaning rod featuring a distal cleaning tip, wherein said rod is sealingly inserted through said proximal seal and through said peripheral conduit, said rod extending longitudinally through said adaptor, and operational to allow insertion and retraction of said rod for physical break up and grinding of fecal debris and removal of clogging matter.

6. The adaptor according to claim 1, wherein said endoscope bore is in fluid communication with said suction port, and wherein said endoscope bore comprises helical grooves configured to promote vortex flow of lumen fluids.

7. The adaptor according to claim 1, wherein said external body-recliner comprises at least one of:
a cushion connected to resilient members;
a cushion connected to a ratchet featuring a toothed bar which is movable with respect to a snapping tooth-stopper;
an external inflatable balloon; and
a pulling strap fixed to said clutch, wherein said clutch is mobile along the length of said muzzle,
for allowing adjusting of the longitudinal position of said clutch in relation to the lumen.

8. The adaptor according to claim 1, further comprising a lumen fluid suction unit in fluid communication with said suction port, for evacuation of fluids from the lumen, and an irrigation unit in fluid communication with the lumen, for streaming irrigation fluids into the lumen.

9. The adaptor according to claim 8, further comprising an external fluid controller for controlling the operation of said lumen fluid suction unit and said irrigation unit, said external fluid controller comprising measuring sensors for measuring fluid flow, vacuum level at said suction port, and/or estimating the amount of cleansing fluid inside the patient's lumen.

10. The adaptor according to claim 9, wherein said lumen fluid suction unit comprises a sewage canister for collecting evacuated lumen fluids,
wherein said irrigation unit comprises an irrigation canister from which irrigation fluids are streamed into the lumen,
and wherein said measuring sensors comprise weighing scales for measuring fluids quantity inside said sewage canister and/or said irrigation canister, allowing to estimate the amount of cleansing fluid inside the patient's lumen.

11. The adaptor according to claim 10, wherein said lumen fluid suction unit further comprises an air tube in fluid communication with an evacuation tube communicating said lumen suction unit with said lumen; and said measuring sensors comprise a pressure sensor configured to measure the pressure level in said evacuation tube,
wherein when the pressure level in said evacuation tube drops below a predefined threshold, said controller controls air streaming means for streaming air into said evacuation tube via said air tube, for raising the air pressure in said evacuation tube without halting the operation of said lumen fluid suction unit.

12. Method for lumen fluid conducting and endoscope coupling, comprising the procedures of:
a) disposing an endoscope into an adaptor, wherein said adaptor comprises a longitudinally extending endoscope bore bored through said adaptor, and a proximal seal configured to surround the endoscope inserted in said bore, effecting blocking of fluid leakage from the proximal end of said adaptor;
b) inserting the distal end of said adaptor into the proximal portion of the patient lumen, said adaptor comprising a distal intra-lumen collapsible clutch, an interim protruding muzzle and a proximal external body recliner, and continuing insertion of said adaptor until said external body recliner interfaces the patient body, wherein a distal lumen seal is operative to block leakage from a gap between the distal end of said muzzle and said lumen;
c) expanding said distal collapsible clutch within said proximal portion of the lumen into an expanded configuration which presses against the lumen to fixate said adaptor to the body of the patient;
d) allowing lumen fluids to drain through a longitudinally extending peripheral conduit bored along said adaptor through said muzzle, and to be released through a fluid suction port which is in fluid communication with said peripheral conduit, said lumen fluids comprising liquids or liquids combined with solids, wherein said proximal seal blocks the fluids from leaking from the proximal end of said adaptor; and
(e) spurting cleansing fluid through said adaptor from an irrigation nozzle.

13. The method according to claim 12, wherein said fixating comprises at least one selected from the group consisting of:
inflating a distal inflatable balloon fixed to said clutch, wherein said inflating of said balloon expands said collapsible intra-lumen clutch into said expanded state; and
inflating an internal balloon connected to said clutch through an inflating tube in fluid communication with said balloon, wherein the tip of which tube remains external to the lumen following said inserting, for allowing external access for inflating and deflating said balloon.

14. The method according to claim 12, wherein said lumen sealing comprises at least one selected from the group consisting of:
inflating of a distal inflatable torus balloon, wherein said torus balloon is fixed to said clutch and is operational when inflated to expand said clutch into said expanded state and to prevent backflow of said lumen fluids by sealing the gap between said protruding muzzle and said lumen when said adaptor is inserted within the lumen; and
expanding a distal flapping peripheral flange operational as said lumen seal, wherein said flange is operational when in said expanded state to prevent backflow of said lumen fluids by sealing the gap between said muzzle and the lumen when said adaptor is inserted within the lumen.

15. The method according to claim 12, wherein said draining of the lumen comprises physically breaking up and grinding of fecal debris and other clogging matter using a cleaning rod featuring a distal cleaning tip, said rod sealingly inserted through said proximal seal and said peripheral conduit, wherein said grinding comprises inserting and retracting of said rod.

16. The method according to claim 15, wherein said draining of the lumen comprises at least one of: (1) spurting cleansing fluid from an irrigation nozzle extending through at least one hollow arm, said hollow arm extending along the length of said cleaning rod, when said irrigation nozzle is inserted into the lumen; and (2) monitoring said grinding and said draining of the lumen using a camera inserted into the lumen through said at least one hollow arm.

17. The method according to claim 12, further comprising adjusting the longitudinal dimensions of said adaptor to fit the proximal potion of the lumen by at least one of:
leaning a proximal external cushion connected to resilient members upon the patient's body;
leaning a proximal external cushion connected to a ratchet featuring a toothed bar which is movable with respect to a snapping tooth-stopper upon the patient's body;
leaning a proximal external inflatable balloon with a regulated degree of inflation upon the patient's body; and/or
pulling said clutch along said muzzle with a pulling strap fixed to said clutch wherein said clutch is mobile along the length of said muzzle.

18. The method according to claim 12, wherein said draining of the lumen comprises: actively suctioning lumen fluids through said suction port; irrigating the lumen with irrigation fluids; and controlling said suctioning and said irrigating by an external fluid controller.

19. The method according to claim 12, further comprising at least one of:
measuring, calculating and/or controlling fluid flow and/or vacuum level at said suction port, and/or
estimating the amount of cleansing fluid inside the lumen of the patient.

20. The adaptor according to claim 1, further comprising a lumen protecting element, at least partially encircling a distal portion of said adaptor, and configured to hinder a wall of the lumen from entering into a cavity at the distal end of said adaptor.
